(19) United States
(12) Patent Application Publication
(10) Pub. No.: US 2023/0347071 A1
(43) Pub. Date: Nov. 2, 2023

(54) Needle Shield Removal Assembly, Method for Assembling Thereof and Drug Delivery Device

(71) Applicant: Sanofi, Paris (FR)

(72) Inventors: Uwe Dasbach, Frankfurt am Main (DE); Robin Einwächter, Frankfurt am Main (DE); Phillip Hintermeier, Frankfurt am Main (DE); Ulrik Michael Jakobi, Frankfurt am Main (DE); Ngoc Jane Lam, Frankfurt am Main (DE); Raik Reimer, Frankfurt am Main (DE)

(21) Appl. No.: 18/013,769

(22) PCT Filed: Jul. 1, 2021
(86) PCT No.: PCT/EP2021/068145

§ 371 Date: Dec. 29, 2022

(30) Foreign Application Priority Data
Jul. 1, 2020 (EP) 20315334.1

(51) Int. Cl. A61M 5/32; A61M 5/20

(52) U.S. Cl. CPC A61M 5/3213; A61M 5/2033; A61M 5/3204; A61M 2205/276

(57) ABSTRACT

A needle shield removal assembly includes a needle shield gripper configured to operatively engage a needle shield, and a gripper holder, wherein the needle shield gripper is secured to the gripper holder via a connection interface, wherein the connection interface is formed by a holder interface feature engaging a gripper interface feature, wherein the gripper holder has an opening configured to receive the needle shield gripper and/or the needle shield, wherein the holder interface feature is offset from the opening, wherein the gripper holder defines a receiving space within which the needle shield gripper is received, wherein the receiving space has an axial extension, and wherein the needle shield gripper and the gripper holder are adjusted to one another to reduce mechanical contact of a) the gripper holder and a bent region of the needle shield gripper and/or b) the gripper holder and an edge of the needle shield gripper.

### CROSS REFERENCE TO RELATED APPLICATIONS

The present application is the national stage entry of International Patent Application No. PCT/EP2021/068145, filed on Jul. 1, 2021, and claims priority to Application No. EP 20315334.1, filed on Jul. 1, 2020, the disclosures of which are incorporated herein by reference.

### TECHNICAL FIELD

The present disclosure relates to a needle shield removal assembly, a method of assembling such an assembly and a drug delivery device.

### BACKGROUND

In drug delivery devices, sometimes, a needle shield covering a needle has to be removed from the needle before a drug delivery operation is performed with the device. This is sometimes done via needle shield removal devices or assemblies. Such removal devices or removal assemblies may facilitate removal of the needle shield for example because removing the needle shield may require considerable forces or because the needle shield is not easily accessible from the exterior of the device.

### SUMMARY

Certain aspects of the present disclosure can provide improvements associated with needle removal assemblies.

One aspect of the present disclosure relates to a needle shield removal assembly for a drug delivery device. Another aspect of the present disclosure relates to a drug delivery device including the needle shield removal assembly. Yet another aspect of the present disclosure relates to a method of assembling a needle shield removal assembly.

The needle shield removal assembly manufactured or assembled in the method is, preferably, the needle shield removal assembly for the drug delivery device. The same holds for the needle shield removal assembly of the drug delivery device. Accordingly, features disclosed with respect to the needle shield removal assembly do also apply for the method and/or the drug delivery device, and vice versa.

In an embodiment, the needle shield removal assembly includes a needle shield gripper. The needle shield gripper may be configured to operatively engage a needle shield, e.g., a needle shield of the drug delivery device. The needle shield may cover a needle of the drug delivery device at least partly. The needle shield gripper may be configured to interlock with the needle shield. The needle shield gripper may be configured to establish a form-fit connection with the needle shield. For example, the needle shield gripper may be configured to dig into an outer surface of the needle shield to establish a reliable connection to the needle shield such that the needle shield may be removed together with the needle shield gripper. The needle shield gripper may be designed to receive the needle shield and/or the needle in an interior of the needle shield gripper.

In an embodiment, the needle shield removal assembly includes a gripper holder. The needle shield gripper may be secured to the gripper holder, e.g., via a connection interface established between the gripper holder and the needle shield gripper. The connection interface may secure the needle shield gripper against movement relative to the gripper holder in at least one direction, preferably an axial direction. The needle shield gripper may be axially and/or rotationally locked to the gripper holder, preferably permanently. The needle shield gripper may be secured in two directions against axial movement and/or rotational movement relative to the gripper holder. The connection interface may be formed by at least one holder interface feature of the gripper holder, preferably by a plurality of holder interface features of the gripper holder, engaging at least one gripper interface feature, preferably a plurality of gripper interface features of the needle shield gripper. If a plurality of interface features are provided, one holder interface feature may engage one associated gripper interface feature. The needle shield gripper may be received in a tube-like section of the gripper holder. An outer surface of the needle shield gripper may face an inner surface of the gripper holder, e.g., of the tube-like section. Of course, different arrangements are possible. However, this arrangement has proven to be particularly advantageous.

In an embodiment, an outer surface of the needle shield gripper abuts an inner surface of the gripper holder. The respective surface may be a radially facing surface.

In an embodiment, the gripper holder has an opening. In the following, this opening is sometimes also referenced as “holder opening”. The opening may be configured and dimensioned to receive the needle shield gripper and/or the needle shield. The (respective) holder interface feature may be offset from the opening, e.g., in a direction away from the opening. A surface of the (respective) holder interface feature, which surface faces away from the opening, and a surface of the gripper interface feature facing towards the opening may be arranged to abut each other to form the connection interface. The holder interface feature may be axially offset from the opening of the gripper holder.

In an embodiment, the gripper holder has or defines a receiving space. In the receiving space, the needle shield gripper may be received and/or arranged. The needle shield gripper may be secured within the receiving space. The receiving space may have an axial extension. The opening may communicate with the receiving space. Accordingly, via the opening the needle shield gripper may be inserted or introduced into the receiving space. During insertion, the gripper interface feature may be moved towards the holder interface feature until the connection interface is established. The axial extension may be the extension away from the opening, e.g., in a direction perpendicular relative to a plane defining the opening. The axial direction may run along an axis running through a center of the opening and/or running perpendicular to the plane defining the opening. At a side remote from the opening the receiving space may be delimited by an abutment surface, e.g., of a surface of the gripper holder.

In an embodiment, the drug delivery device includes, preferably in addition to the needle shield removal assembly, a reservoir. The reservoir may include a drug or medicament, preferably in liquid form. The drug delivery device may include a needle. The needle may be at least partly covered by the needle shield. The needle shield gripper may engage the needle shield, e.g., a radially facing and/or outer surface of the needle shield. The needle shield may be removed from the needle by means of the needle shield removal assembly. That is to say, when the gripper holder is removed, the needle shield is removed as well on account of the connection or interlock between the needle shield gripper and the needle shield. The needle shield may be of plastic. The reservoir may be a cartridge, preferably without a needle, or a syringe, preferably provided with a needle. In order to provide fluid connection between an interior of the cartridge and the needle, the cartridge may be moved relative to the needle during an injection operation or vice versa. This relative movement between needle and reservoir may be avoided, in case a pre-filled syringe is provided. The reservoir may be static during operation of the device or move. Often, the needle and also the needle shield fitted over the needle are usually stored together with the remainder of the device, e.g., the reservoir, such as a syringe barrel. Storage often takes place at low temperatures. Storage at low temperatures tends to increase the adhesion of the needle shield at the reservoir and/or the needle. This accordingly increases the removal forces required to remove the needle shield. Therefore, high removal forces may particularly occur for pre-filled syringes. The present disclosure is, however, not restricted to pre-filled syringes.

In an embodiment, the needle shield gripper has an annular configuration or shape. Thus, the needle shield gripper may resemble a sleeve. The needle shield gripper may be a sleeve or may be different from the sleeve as will be explained further below. A sleeve is a component which does not have free angular ends but only axial ends. The proposed needle shield gripper may have free angular ends as will become apparent from the description further below. However, the present disclosure does also apply for needle shield grippers which have a sleeve shape except where expressly stated.

During investigations of systems of gripper holders and needle shield grippers which have been assembled to each other to form needle shield removal assemblies, it was found that many systems showed damages of one of the components, usually of the gripper holder. Specifically, the systems had scrapes or flakes, e.g., of gripper holder material, e.g., within the receiving space. Of course, having loose particles like scrapes or flakes or noticeable damages in the system may be irritating for a user and may reduce the user's confidence that the device is constructed properly. Alternatively or additionally, removal of material from prefabricated components, e.g., from the gripper holder due to scraping, may decrease the structural stability of the component, and, hence, be problematic, particularly as the forces involved in the removal of the needle shield may be rather high and it should be ensured that the needle shield is reliably removed. Hence, the generation of scrapes or flakes should be avoided or at least reduced. The present disclosure contains various concepts which are suitable to address this problem.

In an embodiment, the needle shield gripper and the gripper holder are configured and/or adjusted to one another to reduce or prevent mechanical contact of the gripper holder and the needle shield gripper, e.g., of one or more sections of the needle shield gripper, such as sections which are particularly likely to cause damages to the gripper holder, and/or one or more regions of the gripper holder. The regions of the gripper holder may be regions which have been identified to be particularly sensitive for being damaged by the needle shield gripper. Therefore, these regions of the gripper holder are also designated as sensitive regions herein. The damages were found to occur most likely during the assembling of the needle shield gripper to the gripper holder, in particular while the needle shield gripper was guided or inserted into the receiving space. Therefore, the needle shield gripper and the gripper holder may be configured and/or adjusted to one another to reduce or prevent mechanical contact of the gripper holder and the needle shield gripper, preferably during insertion of the needle shield gripper into the receiving space via the opening and/or before the connection interface has been established.

In an embodiment, at least one sensitive region of the gripper holder is angularly offset from the at least one holder interface feature. Every sensitive region may be angularly offset from an associated holder interface feature. The number of sensitive regions may be equal to the number of holder interface features or different. Surprisingly, it has been found that regions angularly offset from the holder interface feature were particularly likely to be damaged, especially during the assembling of the needle shield removal assembly. The angular position of the sensitive regions may be angularly in the middle between two adjacent holder interface features. Accordingly, preventing or reducing contact in these region is particularly advantageous.

In an embodiment, the at least one sensitive region is arranged angularly between two angularly adjacent holder interface features.

In an embodiment, the respective sensitive region is a region of an inner surface of the gripper holder.

In an embodiment, the section of the needle shield gripper of which contact with the gripper holder may be reduced or prevented, e.g., in the sensitive region(s), is a bent or kinked region of the needle shield gripper and/or an edge or corner of the needle shield gripper. It has been found that a kinked or bent regions of the needle shield gripper are particularly likely to damage the gripper holder, e.g., by generating scrapes or flakes, when engaging the gripper holder, e.g., in the region angularly offset from the holder interface feature. Alternatively or additionally, the same holds for edges or corners of the needle shield gripper, particularly in a region angularly offset from the holder interface feature. In the context of the present disclosure, “angular” may refer to the azimuthal direction, i.e., the direction defined by the azimuthal angle or rotation angle relative to an axis, e.g., relative to the longitudinal axis running through the opening.

The edge or corner may be arranged at an end of the needle shield gripper, e.g., at a leading end. The end may face in the axial direction. The leading end may be the end which is introduced at first into the opening and/or the end which is further away from the opening in the needle shield removal assembly. The edge or corner may refer to a surface region of the needle shield gripper which faces away from the opening. The bent or kinked region may extend along the side surface of the needle shield gripper. The bent or kinked region may define the edge or corner, e.g., in an end region thereof, or not. The edge or corner may be part of a sharp section of the needle shield gripper. The edge or corner may include an angled section, e.g., at the end of the bent or kinked region. The angle defined by the angled section may be arranged in a plane perpendicular to the axis. The corner may be an angled section of the edge of the needle shield gripper, e.g., at a leading end of the needle shield gripper.

Edges or corners at an end of the needle shield gripper have proven to be particularly likely to generate flakes or scrapes during the assembling process, in particular when an angled section contacts the gripper holder. The edge or corner may be defined by punching, or another method which generates a sharp edge. Also, bent or kinked regions of the needle shield gripper, e.g., on a lateral side surface, especially in an end region of the side surface close to the leading end, may contribute significantly to the damages or cause the majority thereof. Therefore, it is particularly advantageous to reduce or prevent contact of these sections of the needle shield gripper with the gripper holder in a sensitive region. In non-sensitive regions there may be mechanical contact during and/or after the insertion of the needle shield gripper into the receiving space.

In an embodiment, the needle shield gripper has a plurality of kinked or bent regions and/or corners or edges. One kinked or bent region may separate two plane surfaces of the needle shield gripper. Not all of the kinked or bent regions and/or the edges or corners may be angularly aligned with the sensitive region. The sections of the needle shield gripper which are angularly aligned with sensitive regions are the ones which are preferably adjusted or modified to reduce or prevent mechanical contact as opposed to other sections, which may have the same configuration as the sections angularly overlapping with the sensitive regions before they are adjusted or modified. Alternatively or additionally, the sections of the gripper holder which are adjacent to or within the sensitive region(s) may be adjusted or modified to reduce or prevent mechanical contact between the gripper holder and the needle shield gripper in the sensitive region. Surprisingly, it has been found that a region of the gripper holder which is angularly offset from the holder interface feature is particularly likely to be engaged with the needle shield gripper during insertion and to generate scrapes or flakes due to mechanical cooperation with the needle shield gripper. Accordingly, by adjusting the needle shield gripper and the gripper holder such that mechanical contact is prevented or reduced in that particular region, generation of scrapes or flakes can be reduced or even entirely prevented. This increases the user's confidence that the device is working properly, for example.

In an embodiment, the gripper holder includes a plurality of holder interface features. The holder interface features are expediently aligned axially, i.e., at the same axial positons, and/or angularly separated from one another. Preferably, each holder interface feature has one corresponding gripper interface feature. The holder interface features may be evenly distributed in the circumferential or angular direction. That is to say the distance between two angularly adjacent holder interface features may be equal, preferably independent of which pair of angularly adjacent holder interface features is chosen.

In an embodiment, the beginning of one or more sensitive regions of the gripper holder may be defined by the axial position of the holder interface feature. Alternatively or additionally, the sensitive region may be distally offset or offset away from the opening relative to the holder interface feature(s).

In an embodiment, the angular distance between one holder interface feature of the gripper holder and the sensitive region is less than the distance between two adjacent holder interface features, preferably less than or equal to half the distance. In other words, one sensitive region may be provided between two angularly adjacent holder interface features, e.g., in the middle.

In an embodiment, the respective holder interface feature protrudes, preferably inwardly, into the receiving space. Particularly, the holder interface feature(s) may define a free diameter in a region of the receiving space. In the region of the holder interface feature(s), the inner diameter of the receiving space may be less than the outer diameter of the needle shield gripper, particularly in a section of the needle shield gripper adjoining the holder interface feature in the direction towards the opening and/or the direction away from the opening.

In an embodiment, the needle shield gripper is deformable, preferably elastically deformable. The needle shield gripper may be deformable in the radial direction. That is to say when a radially directed force is applied to the needle shield gripper, the needle shield gripper may be deformed and, once the force is removed, may resume its original shape due to the elastic restoring force. This may be advantageous during assembling, as the elastic deformation may be required for engaging the holder interface feature and the gripper interface feature. However, if the needle shield gripper is elastically deformed, the shape, e.g., its cross section, may change and, consequently, the regions of the gripper holder engaged by the deformed needle shield gripper may change and/or the force acting on the regions engaged by the deformed needle shield gripper may be increased. The respective sensitive region may be a region into or towards which the needle shield gripper is deformed during the assembling process, in particular when and/or after the needle shield gripper engages the holder interface feature(s) and/or until the gripper interface feature has engaged the holder interface feature to establish the connection interface. The needle shield gripper may be less elastically deformable or even rigid when exposed to axially directed forces, i.e., forces which act along the axial direction, such as along the axial extension of the kinked or bent region(s) of the needle shield gripper. Accordingly, the elastical deformability in the radial direction may be used to assemble the needle shield gripper and the gripper holder to one another. The undeformed or relaxed needle shield gripper may have an outer diameter which is greater than the inner diameter defined in the region of the holder interface features, greater than the inner diameter defined in a region offset from the holder interface feature(s) towards the opening and/or greater than the inner diameter defined in region offset from the holder interface feature(s) away from the opening. That is to say, when the needle shield gripper has been introduced into the opening, an elastic restoring force may act to maintain an abutment between an inner surface of the gripper holder and an outer surface of the needle shield gripper.

In an embodiment, the respective holder interface features a snap feature.

In an embodiment, the needle shield gripper includes an oblique surface region, which may be plane. The surface region may be an exterior surface region of the needle shield gripper. As seen from the end of the needle shield gripper closest to the oblique surface region, the oblique surface region may extend radially away from the axis with increasing distance from that end. An angle between the oblique surface region and the axis, e.g., the one defined by the axial extension of the receiving space, may be less than 90°, e.g., less than or equal to one of the following values: 60°, 55°, 50°, or 45°. The oblique surface region may form a leading surface or a lead-in surface of the needle shield gripper which is provided to interact with the holder interface feature in order to elastically deform the needle shield gripper during insertion of the needle shield gripper into the receiving space. The oblique surface region may merge or lead into an axially extending surface region of the needle shield gripper as seen in a direction away from the leading end of the needle shield gripper. The oblique surface region may be angularly aligned with or angularly overlap with the gripper interface feature.

In an embodiment, the oblique surface region angularly overlaps with or is angularly aligned with the at least one holder interface feature, particularly when the gripper holder and the needle shield gripper have been assembled. If a plurality of holder interface features are provided, preferably at least one oblique surface region or only one oblique surface region is provided which may interact with one of the holder interface features during insertion. However, it is also possible that a plurality of oblique surface regions are provided. When the needle shield gripper has been inserted into the receiving space, the oblique surface region may be arranged axially offset from the opening and the holder interface feature.

In an embodiment, the needle shield gripper includes a plurality of bent or kinked regions and/or a plurality of edges or corners. One gripper region of the needle shield gripper, such as a plane region or a less curved or bent region than the kinked or bent region, may be arranged between two adjacent bent or kinked regions. The bents or kinks of the bent or kinked regions may be oriented along one another, e.g., parallel. The respective bent or kinked region may be oriented axially. The respective gripper region may form a region of the side surface of the needle shield gripper, e.g., a plane or curved surface region.

In an embodiment, the at least one sensitive region may be arranged angularly between two adjacent bent or kinked regions and/or between two angularly adjacent edges or corners. In other words, the kinked or bent regions or edges or corners closest to the sensitive region may be angularly offset from the sensitive region. In this way, it can be avoided that the kinked bent regions and/or the edges or corners do contact the gripper holder in the sensitive region. Consequently, flake or scrape generation in the sensitive region can be avoided or at least significantly reduced. The region of the needle shield gripper between the two adjacent bent or kinked regions may be plane, e.g., with a plane outer surface.

In an embodiment the bent or kinked region is arranged angularly within the sensitive region.

In an embodiment, the gripper holder has a plurality of spatially separated sensitive regions or only one sensitive region.

In an embodiment, the sensitive regions are evenly distributed in the circumferential or angular direction.

In an embodiment, the gripper interface feature is axially offset from the leading end of the needle shield gripper.

In an embodiment, at least a portion of the sensitive region is arranged axially offset from the holder interface feature in a direction away from the opening. That is to say, the region between the holder interface feature and an end of the receiving space facing away from the opening may be particularly prone to scraping or flake generation.

In an embodiment, the needle shield gripper has a cutout. The cutout may extend in the axial direction, i.e., the cutout may be an axially extending or oriented cutout. The cutout may extend from an end, e.g., from the leading end, of the needle shield gripper, e.g., towards the opening. The cutout may be provided in the leading end region of the needle shield gripper. The cutout may have a width. The width of the cutout may reduce along the axial extension of the cutout, e.g., when seen in the direction away from an end of the needle shield gripper. In other words, the cutout may taper, e.g., towards the opening. An end surface of the cutout may run perpendicularly relative to the axis. Side surfaces of the cutout may run obliquely with respect to the axis, that is to say, neither parallel nor perpendicular. An angle which the oblique surfaces define with the axis may be less than 90°, e.g., less than or equal to 60° such as less than or equal to 45°.

In an embodiment, one kinked or bent region and/or the corner or edge may overlap angularly with the cutout. An end of the kinked or bent region and/or the corner or edge, may be arranged in the cutout. The kinked or bent region and/or the corner or edge may be angularly offset from an angular center point of the cutout. The angular center point may be arranged in that region of the cutout which runs perpendicularly with respect to the axis. The kinked or bent region may define the angular border between the perpendicular section of the surface of the cutout and the oblique surface of the cutout. Arranging the kinked or bent region offset from the center point and/or offset from the perpendicular surface region may assist in preventing the generation of flakes and/or scrapes.

In an embodiment, the needle shield gripper is elastically deformed when the connection interface has been established. In other words, there may still be an elastic deformation when the connection interface is established. This ensures that an inner surface of the gripper holder and an outer surface of the needle shield gripper are maintained in contact and the connection between the needle shield gripper and the gripper holder is particularly reliable.

In an embodiment, the outer diameter of the needle shield gripper in a non-deformed state, i.e., when the needle shield gripper is not elastically deformed, is greater than the inner diameter of the opening. Thus, in order to introduce the needle shield gripper into the opening the needle shield gripper has to be deformed elastically. This elastic deformation may occur prior to an engagement of the holder interaction feature with the outer surface of the needle shield gripper. The engagement may increase the elastic radial deformation, e.g., on account of the reduced diameter in the region of the holder interface feature(s). However, the initial elastic deformation may already retain the needle shield gripper in a defined position relative to the gripper holder. This is, of course, advantageous for the assembling process.

In an embodiment, a cross section of the needle shield gripper in a relaxed state, i.e., when it is not elastically deformed, or non-deformed state is different from a cross-section of the opening and/or the receiving space in the elastically deformed state when it is arranged in the receiving space, e.g., the cross sections have different shapes. The receiving space may, if applicable aside from the region where the holder interface feature(s) is(are) provided, have a circular shape or cross section.

In an embodiment, in a region angularly offset from the gripper interface feature(s), the diameter, e.g., the inner and/or outer diameter, of the needle shield gripper may be less than in the region of the gripper interface features in the relaxed state, e.g., when the needle shield gripper is not arranged in the receiving space and/or not deformed elastically. The region with the smaller diameter may be a region which is configured to be angularly aligned with the sensitive region when the needle shield gripper has been assembled to the gripper holder. The region may be angularly aligned with the sensitive region in the needle shield removal assembly. Therefore, when deformation of the needle shield gripper occurs during the assembling process, the difference in diameter has to be compensated before the sensitive region, which may be likewise angularly offset from the gripper interface features when the gripper holder and the needle shield gripper have been assembled, is contacted. The cross section of the needle shield gripper in the relaxed state may be oval or polygonal.

In an embodiment, the needle shield gripper has at least one needle shield interlock feature which is adapted to interlock with the needle shield. The needle shield gripper may have a plurality of needle shield interlock features. The needle shield interlock features may be evenly or unevenly distributed in the angular direction. The needle shield interlock features may be axially aligned. The respective holder interface feature may be angularly aligned with an associated needle shield interlock feature. This has an advantage, as the needle shield interlock features may be generated by forming an opening within the needle shield gripper. The opening in the needle shield gripper may be used as a gripper interface feature to engage an associated holder interface feature. Thus, formation of a separate gripper interface feature in the needle shield gripper can be avoided. Of course, it is also possible to form separate gripper interface features. Accordingly, having the at least one holder interface feature angularly aligned with an associated needle shield interlock feature may be advantageous. The respective needle shield interlock feature may be a barb. The respective needle shield interlock feature may protrude radially inwardly.

In an embodiment, the needle shield gripper has two free angular ends. As opposed to a closed annular configuration of the needle shield gripper without free ends, such a configuration with two free angular ends may be advantageous from a manufacturing perspective. Then the needle shield gripper may be formed from a unitary sheet by bending. The angular ends may overlap such that the needle shield gripper defines a circumferentially closed interior space, which is preferably adjusted to receive the needle shield. In an alternative embodiment, the needle shield gripper has an annular configuration.

In an embodiment, the respective bent or kinked region extends axially, preferably along the entire needle shield gripper, that is to say from a first end towards and preferably up to a second end, where one of these ends may be the leading end.

In an embodiment, the needle shield gripper, preferably in an end section of the bent or kinked region, includes an indentation. The indentation may be radially, particularly radially inward, directed. The indentation may be arranged to face the at least one sensitive region when the needle shield gripper has been introduced into the receiving space. By indenting the needle shield gripper in the region which is to be disposed to face the sensitive region an interaction between the potentially sharp edge or corner region of the needle shield gripper and a (inner) surface of the gripper holder may be avoided or reduced. The indentation may adjoin the bent or kinked region and performed by indenting the bent or kinked region after bending or kinking has been performed. Due to the indentation, contact in the sensitive region may be avoided or at least reduced.

In an embodiment, the needle shield gripper includes a tab. The tab may be arranged between the bent or kinked region and a (inner) wall of the gripper holder, in particular between the bent or kinked region and the sensitive region. Thus, the tab may prevent contact of the gripper holder and the bent or kinked region in the sensitive region of the gripper holder. In an end section of the needle shield gripper, e.g., a section close to the leading end, the bent or kinked region may be covered by the tab. The tab may be arranged between the needle shield gripper and the gripper holder during the insertion and/or when the connection interface has been established. The tab may be arranged at a radial position between an inner wall of the gripper holder and the bent or kinked region. The tab may be moved to a position covering the bent or kinked region after the bending process to define the bent or kinked region(s) has been performed. The (outer) surface of the tab facing the (inner) wall of the gripper holder may be less bent or kinked than the bent or kinked region.

In an embodiment, the needle shield gripper is of a unitary structure.

In an embodiment, one of the gripper holder and the needle shield gripper has at least one protrusion or a plurality of protrusions. The respective protrusion may be arranged to abut or abuts the other one of the gripper holder and the needle shield gripper. The protrusions may be evenly distributed in the angular direction or non-evenly distributed, i.e., localized. Alternatively or additionally, the protrusions may be associated with or may be disposed to be arranged close to, e.g., angularly close to, the (respective) sensitive region. Alternatively or additionally, the respective protrusion may be associated with a bent or kinked region and/or an edge or corner. Preferably, a plurality of protrusions may be associated with the bent or kinked region and/or the edge or corner. The bent or kinked region may be disposed angularly between the protrusions. By way of the protrusions, it can be avoided that contact is made between the needle shield gripper and the gripper holder in the sensitive region, as the contact may be prevented by abutment with the respective protrusion which prevents mechanical contact of the needle shield gripper and the gripper holder in the sensitive region. The respective protrusion may be angularly disposed outside of the sensitive region and/or delimit the sensitive region angularly.

In an embodiment, the respective protrusion protrudes radially, e.g., inward or outward. The respective protrusion may have a radial free end.

In an embodiment, the needle shield gripper includes the protrusion(s). The protrusion may be arranged to engage or engages the gripper holder, preferably an inner surface of the gripper holder.

In an embodiment, the protrusion is associated with the bent or kinked region of the needle shield gripper and/or the edge or corner of the needle shield gripper. The protrusion may maintain a defined relative radial position, preferably with a radial spacing, between the bent or kinked region of the needle shield gripper and/or the edge or corner of the needle shield gripper and the gripper holder, e.g., during the assembling and/or when the needle shield gripper has been introduced into the receiving space. Preferably, at least two protrusions are associated with one bent or kinked region. The protrusions may be axially aligned. The bent or kinked region may be arranged angularly between the at least two protrusions. Alternatively or additionally a corner or angled region of the leading end or edge of the needle shield gripper may be arranged angularly between the at least two protrusions. If there is a plurality of bent or kinked regions which could potentially be arranged to face a sensitive region during the assembly process or in the assembled needle shield removal assembly, each of these bent or kinked regions or, as the case may be, edges or corners may have associated protrusions.

In an embodiment, the free end of the protrusion may be radially and/or angularly offset from a radial end of the bent or kinked region with which the protrusion is associated. The free end of the protrusion may protrude radially beyond the bent or kinked region and/or the edge or corner of the needle shield gripper. The needle shield gripper may include at least two protrusions associated with the bent or kinked region of the needle shield gripper and/or the edge or corner of the needle shield gripper. The free ends of the protrusions may be radially offset from the radial end of the bent or kinked region and/or the edge or corner of the needle shield gripper with which the protrusions are associated. An enveloping curve running through free ends of the protrusions and extending along the outer circumference of the needle shield gripper, e.g., a circle, may be radially offset from the radial end of the bent or kinked region and/or of the edge or corner.

In an embodiment, the protrusion may be formed as a bulge of the needle shield gripper. The bulge may be formed by an indentation of the surface of the needle shield gripper opposite from the bulge, e.g., formed by deep-drawing or embossing. This facilitates formation of the bulge during production and does not require too many modifications in the production process for the needle shield gripper. The bulge may be radially oriented.

In an embodiment, the protrusion has a circumferentially closed boundary, e.g., a circular boundary, on the surface of the needle shield gripper from which the protrusion protrudes.

In an embodiment, the protrusion is formed by an axially extending rib of the gripper holder. The rib may have a radial free end. The length of the rib may be adjusted to the axial extension of the needle shield gripper, e.g., at least 70% of the length of the needle shield gripper. In this way, the protrusion may reliably prevent the mechanical contact of the needle shield gripper and the gripper holder.

In an embodiment, the at least one protrusion is defined by molding, embossing, or deep drawing. These processes can be easily integrated into the production of the gripper holder and/or the needle shield gripper, respectively, where embossing or deep drawing is particularly suitable for the needle shield gripper, which may be of metal, and molding is particularly suitable for the gripper holder, which may be of plastic.

In an embodiment, the radial extension of the respective protrusion is greater than or equal to one of the following values: 0.1 mm, 0.2 mm, 0.3 mm. Alternatively or additionally, the radial extension of the protrusion may be less than or equal to one of the following values: 2 mm, 1.5 mm, 1 mm, 0.7 mm, 0.6 mm, 0.5 mm, 0.4 mm. Ranges may be formed by combining values specified above as boundaries.

In an embodiment, the protrusion, e.g., at least a portion of the protrusion or the entire protrusion, is axially offset from the at least one holder interface feature in a direction away from the opening. In this region of the gripper holder, scraping and flakes were found to occur with a high likelihood.

In an embodiment, the needle shield gripper has at least one gripper guide feature, for example a cutout, such as the cutout discussed further above. The gripper guide feature may be arranged to cooperate with a holder guide feature, e.g., by abutment. The holder guide feature may be provided in the gripper holder. The mechanical cooperation of the holder and gripper guide features may establish a guiding interface in order to achieve a desired angular relative orientation of the needle shield gripper and the gripper holder. In this way, it may be achieved that a holder interface feature and an associated gripper interface feature can be reliably brought into engagement during insertion of the needle shield gripper into the receiving space. The mechanical cooperation, e.g., an abutment, of the holder guide feature and the gripper guide feature may be configured to guide the holder interface feature and the gripper interface feature into engagement.

In an embodiment, the gripper holder includes a radially extending recess or radial recess or a radial opening, particularly a radially extending opening. The recess or the opening may be arranged in the sensitive region, e.g., to prevent or reduce mechanical contact with the needle shield gripper in the sensitive region. Accordingly, by removing material from the gripper holder in the sensitive region, the generation of flakes or scrapes can be avoided or reduced. The recess or radial opening or at least a section thereof may be offset from the holder interface feature(s) in a direction away from the opening, e.g., the holder opening.

In an embodiment, the radial recess or radial opening is angularly offset from the at least one holder interface feature. Alternatively or additionally, the radial recess or radial opening is axially offset from the at least one holder interface feature. At least a portion of the radial recess or opening may be arranged in a section of the gripper holder which is further away from the holder opening than the holder interface feature, e.g., an end surface thereof facing away from the holder opening. The radial recess or radial opening may be provided in the respective sensitive region which is further discussed above and also below. Accordingly, features which are described with reference to the sensitive region should also be regarded as referring to the radial recess or opening.

In an embodiment, the receiving space is, preferably laterally and/or circumferentially, delimited by a portion of the gripper holder. The portion of the gripper holder may have a tubular or cylindrical configuration. Specifically, the portion may include at least one section with a cylindrical surface, preferably closed in the angular direction. The section may be provided between the holder opening and the at least one holder interface feature.

In an embodiment, the portion of the gripper holder which delimits the receiving space is radially inwardly offset from an inner surface of the gripper holder, particularly from an inner surface of an outer wall of the gripper holder. Specifically, a free space or hollow may be formed between an outer surface of the portion of the gripper holder defining the receiving space and an inner surface of the gripper holder, e.g., an inner surface of the outer wall. In this way, the receiving space may be formed internally within the gripper holder and be less prone to forces impacting on the outer wall. The outer wall of the gripper holder may provide a user interaction surface, particularly an external surface of the gripper holder, which can be gripped by the user, e.g., in order to remove the needle shield. An outer surface of the portion delimiting the receiving space may face the inner surface of the outer wall, particularly with no further surface(s) in between.

In an embodiment, the radial recess or radial opening is connected to the receiving space. The radial recess or radial opening may extend outwardly, particularly radially outwardly, as seen from the receiving space. The radial recess or radial opening may extend into or through the gripper holder or a portion thereof. Expediently, the portion through which or into which the recess or opening extends is the portion delimiting or defining the receiving space.

The recess may be radially delimited by material of the gripper holder, e.g., by the portion delimiting the receiving space. The opening may radially protrude through the entire portion and/or the entire gripper holder as the case may be. The opening may connect the receiving space with the free space or hollow defined between the outer surface of the portion of the gripper holder defining the receiving space and the inner surface of the gripper holder mentioned above.

In an embodiment, the radial recess or radial opening includes a first axial end. The first axial end of the radial opening or radial recess may be formed by the portion of the gripper holder defining the receiving space. The first axial end may face towards the holder opening and/or delimit the opening or recess axially on the side of the holder opening. The radial recess or radial opening may extend in the axial direction away from the first axial end. The first axial end may be closed. In other words, an axially oriented surface, e.g., a distally directed surface, may define the first axial end of the radial recess or radial opening. The first axial end may be axially offset from the holder opening, particularly away from the holder opening as seen along the direction of extension of the receiving space away from the holder opening and/or distally. The first axial end may be the proximal end of the radial recess or opening.

In an embodiment, the radial recess or radial opening includes a second axial end. The second axial end may be axially offset from the first axial end and/or from the at least one holder interface feature, e.g., in a direction away from the holder opening and/or distally. The second axial end may be the end remote from the first axial end.

In an embodiment, the second axial end of the radial recess or radial opening is an open axial end. In other words the gripper holder may be free of an axially, e.g., proximally, directed surface which defines the second axial end of the recess or opening. The second axial end may be the distal end of the recess or opening. An open axial end facilitates a definition of the radial opening or radial recess by molding.

In an embodiment, the gripper holder is a unitary component. The gripper holder may be moldable, e.g., in a single step molding process and/or using just one mold.

In an embodiment, a closure, e.g., a lid, is connected to the gripper holder in order to close the gripper holder. The closure may be provided to close the gripper holder on a side which is remote from the holder opening. The closure may form the distal end face of a cap assembly and/or a needle shield removal assembly including the gripper holder and the closure. The second axial end of the respective radial opening or recess may be closed by the closure connected to the gripper holder.

In an embodiment, one or more side surfaces, e.g., angular side surfaces, delimiting the radial opening or radial recess, e.g., angularly, are inclined. The respective side surface may be inclined relative to an axis extending along the longitudinal direction, e.g., from the proximal end to the distal end, of the gripper holder. The side surfaces may face one another. Two angular side surfaces delimiting the radial recess or opening angularly and facing one another in the angular direction may be inclined. The side surfaces may be connected by an end surface arranged at the first axial end of the radial recess or radial opening.

In an embodiment, a width of the radial recess or opening, particularly an angular width, varies as seen along the axial extension of the radial recess or radial opening.

In an embodiment, the angular width of the radial recess or radial opening increases in a direction away from the first axial end, e.g., continuously. That is to say, the opening or recess may widen as seen in a direction away from the first axial end, e.g., continuously. This improves the moldability of the gripper holder including the radial opening or the recess.

In an embodiment, the at least one holder interface feature, e.g., every one of the holder interface features, includes an inclined region or inclined section. The inclined region may be arranged at an end of the holder interface feature facing towards the holder opening and/or proximally. This facilitates an (elastic) deformation of the needle shield gripper when the gripper is introduced into the receiving space on account of mechanical cooperation between the needle shield gripper and the holder interface feature(s). The inclined region or section may form a lead-in region for the cooperation between the needle shield gripper and the gripper holder during insertion of the gripper into the receiving space. The inclined region may be inclined relative to a longitudinal axis of the receiving space. The longitudinal axis may extend from the proximal end to the distal end of the receiving space. The angle of inclination relative to the longitudinal axis may be an acute angle, e.g., an angle less than 45°. The holder interface feature may include a (first) region which is less inclined relative to the longitudinal axis than the inclined region, e.g., the region may extend parallel to the longitudinal axis. This region may be arranged distally to the inclined region. An end region or end surface of the interface feature facing away from the holder opening may be inclined relative to the longitudinal axis with a greater angle than the first region, e.g., the end region or surface may be oriented perpendicular relative to the longitudinal axis. The end region or surface may be provided to interact with the gripper interface feature in order to form the connection interface. In other words, the holder interface feature may include a plurality of regions with different inclinations relative to the longitudinal axis.

In an embodiment, the first axial end of the radial recess or the radial opening is associated with the holder interface feature, particularly with the axial position of the holder interface feature. For example, the first axial end of the radial recess or radial opening may be arranged axially offset from the holder interface feature, e.g., close to the holder interface feature, and/or overlap axially with the holder interface feature. For example, an axial offset between the holder interface feature, particularly an end thereof facing the holder opening, and the first axial end may be less than or equal to anyone of the following values:
the distance and/or axial offset AO between the holder opening and the holder interface feature preferably less than or equal to c*AO, where c is chosen from: 0.5, 0.4, 0.3; and/or
less than or equal to any of the following values: 5 mm, 4 mm, 3 mm, 2 mm; Alternatively or additionally, the axial offset between the holder interface feature, particularly the end thereof facing the holder opening, and the first axial end may be greater than or equal to any of the following values: 0.1 mm, 0.2 mm, 0.3 mm, 0.5 mm, 0.6 mm, 0.7 mm, 0.8 mm, 0.9 mm, 1 mm;

For example, the axial offset may be between 0.1 mm and 5 mm; and/or
the axial extension of the holder interface feature.

In case holder interface features of varying distances to the holder opening are employed the minimum distance between the first axial end and the holder interface features, the maximum distance, or the average distance over the number of holder interface features may be used to determine the axial offset AO between the holder opening and the holder interface features.

Alternatively to a non-zero offset, the axial offset may be zero. If there is no axial offset, the axial end of the radial recess or opening coincides with the onset of the holder interface feature. A non-zero offset may account for tolerances, e.g., during manufacturing, whereas the manufacturing of the gripper holder with zero axial offset may be more cost effective.

In an embodiment, the first axial end of the radial recess or radial opening is arranged axially offset from an end of the at least one holder interface feature, e.g., an end of the at least one holder interface feature facing towards the holder opening. The first axial end may be axially offset from all holder interface features. The first axial end of the radial recess or radial opening may be arranged closer to the holder opening than the end of the (respective) holder interface feature. In other words, the axial offset between the first axial end and the holder opening may be smaller than the axial offset between the end of the holder interface feature facing the holder opening and the holder opening. In this way, it can be guaranteed that the risk of scraping or flake generation is reduced from the moment when the needle shield gripper commences engagement with the holder interface feature.

In an embodiment, the first axial end of the radial recess or radial opening axially coincides with an end of the at least one holder interface feature, e.g., an end of the at least one holder interface feature facing towards the holder opening. This configuration may have a zero axial offset between the first axial end and the end of the at least one holder interface feature.

In an embodiment, the first axial end of the radial recess or radial opening axially overlaps with the at least one holder interface feature. Preferably, the first axial end overlaps axially with the region of the at least one holder interface feature which is inclined, e.g., in the inclined region at that end of the at least one holder interface feature which faces the holder opening. In other words, the axial position of the first axial end may be within the inclined region of the interface feature(s).

In an embodiment, the needle shield gripper includes at least one kinked or bent region which overlaps with the radial opening or radial recess, particularly as seen in the angular direction and/or the axial direction. The angular width of the radial recess or opening is expediently greater than the angular width of the kinked or bent region of the needle shield gripper, e.g., of all kinked or bent regions of the gripper. In this way, it can be ensured that the risk of the needle shield gripper scraping or generating flakes from the material of the gripper holder is reduced considerably. The axial extension of the radial recess or opening may be smaller than the axial extension of the kinked or bent region of the needle shield gripper, e.g., of all kinked or bent regions of the gripper.

In an embodiment, the needle shield gripper protrudes radially into the radial recess or opening. Preferably, there is a radial clearance between the needle shield gripper, particularly the kinked or bent region of the gripper angularly overlapping with the recess, and the gripper holder in the region of the radial opening or recess.

In an embodiment, at least a portion of the kinked or bent region, e.g., only a portion thereof, extends axially along the radial opening. Thus, the kinked or bent region may axially and angularly overlap with the radial recess or opening.

In an embodiment, the gripper holder is of a softer material, e.g., plastic, than the needle shield gripper, which may be of metal. Accordingly, the gripper holder may be more easily scratched than the needle shield gripper.

In an embodiment, the needle shield gripper is of metal. The gripper holder may be of plastic.

In an embodiment, the radial extension of the respective protrusion—e.g., the one on the gripper holder or on the needle shield gripper—is greater than or equal to one of the following values: 0.1 mm, 0.2 mm, 0.3 mm. Alternatively or additionally, the radial extension of the respective protrusion may be less than or equal to one of the following values: 2 mm, 1.5 mm, 1 mm, 0.7 mm, 0.6 mm, 0.5 mm, 0.4 mm. Ranges may be formed by combining values specified above as boundaries.

In an embodiment, the holder interface feature is arranged to block movement of the needle shield gripper towards the holder opening, e.g., proximal movement of the gripper, by a blocking portion of the needle shield gripper abutting a surface of the holder interface feature. The surface of the holder interface feature preferably faces away from the opening, e.g., distally. The blocking portion of the needle shield gripper may face towards the opening, e.g., proximally. The blocking portion may be arranged close to or be associated with an end surface of the needle shield gripper. The blocking portion of the needle shield gripper may be arranged in a region of the needle shield gripper between an end surface of the needle shield gripper and an axially oriented portion of the needle shield gripper. The blocking portion may form or define the surface of the gripper interface feature which is arranged to mechanically cooperate, e.g., to abut, the holder interface feature, such as a distal surface of the holder interface feature.

Consequently, as the blocking portion is provided for mechanical contact with the holder interface feature it can be avoided that the end surface is used to interact with the holder interface feature. Accordingly, mechanical contact between the holder interface feature and an end surface with potentially sharp edges or surface regions may be avoided. Thus, when the gripper is guided into the holder it may be avoided that the end surface mechanically interacts with a surface of the holder interface feature, e.g., a slanted or sloped surface. This reduces the risk of flake or scrape generation in the region of the holder interface feature and the gripper interface feature.

In an embodiment, the end surface faces in a non-axial and/or non-proximal direction. In other words, the end surface of the needle shield gripper may face in a radial direction, e.g., the radially inward direction, or in the distal direction or away from the holder opening. Specifically, the end surface may be a radially oriented surface or a distally oriented surface. The end surface may face in the same direction as the surface of the holder interface feature which is arranged to abut the blocking portion. This, of course, is particularly suitable for a distally facing end surface.

In an embodiment, the end surface is radially offset, e.g., inwardly offset, from the holder interface feature and/or from the blocking portion.

In an embodiment, the end surface is axially, e.g., distally, offset from the blocking portion or axially aligned with the blocking portion.

In an embodiment, the blocking portion is arranged in the region of a tab or tongue of the gripper holder. The tab or tongue may extend strip like from a main body of the gripper holder. A free end of the tab or tongue may define the end surface of the needle shield gripper. The blocking portion may be arranged between that end surface and the axially oriented portion of the needle shield gripper. The tab or tongue may be bent or curved to define the blocking portion.

In an embodiment, the blocking portion is arranged in a curved or bent region of the needle shield gripper. The region may be concavely curved as seen from a distal end of the needle shield gripper.

In an embodiment, the blocking portion is arranged close to the end surface, preferably less than one of the following distances offset from the end surface, preferably as seen along the extension of the tab or tongue away from the end surface: 2 mm, 1 mm, 0.5 mm.

In an embodiment, the blocking portion is a radially extending or oriented portion of the needle shield gripper.

In an embodiment, the end surface is defined by a cutting-off process, e.g., by cutting or punching.

In an embodiment, the end surface is delimited by one or more sharp edges or is provided with a sharp surface structure. The edges of the surface structure may be defined by the production process, e.g., cutting or punching.

In an embodiment, the needle shield gripper is of metal and/or the gripper holder is of plastic.

In an embodiment, the end surface and/or the blocking portion is angularly aligned with the holder interface feature and, preferably, distally offset from the holder interface feature or offset in a direction away from the opening from the holder interface feature.

In an embodiment, the blocking portion is arranged in a transition region of the needle shield gripper. The transition region may be a curved region, e.g., a concavely curved region as seen from that end of the needle shield gripper which is remote from the holder opening, such as from the distal end. In the transition region, the needle shield gripper may change its orientation.

In the transition region, the needle shield gripper may change its orientation from a predominantly axial orientation to a predominantly radial orientation. Specifically, in this manner a radially facing end surface may be formed, e.g., in the course of extension of the needle shield gripper in the proximal direction as seen from the distal end of the needle shield gripper or that end which faces away from the holder opening to the end surface.

In an embodiment, an end of the blocking portion is an end of the needle shield gripper, e.g., a free end. Particularly, the needle shield gripper may be designed such that the blocking portion is immediately adjacent to the end surface.

In an embodiment, the gripper interface feature is an opening which is axially, e.g., distally, delimited by the blocking portion. Hence, the end surface of the opening can be rendered less sharp by providing the blocking portion as an axial end of the opening instead of a sharp or edged end surface.

In an embodiment, the blocking portion is angularly aligned with one of the needle shield interlock features. Alternatively or additionally, at least one of the needle shield interlock features, e.g., the one angularly closest to the blocking portion, may protrude radially, e.g., inwardly, beyond the end surface. The needle shield interlock feature(s) may be axially offset from the blocking portion, e.g., proximally offset.

In an embodiment, the blocking portion is provided in a curved or rounded region of the needle shield gripper.

In an embodiment, the needle shield gripper is concavely curved, preferably as seen from an end of the needle shield gripper which faces away from the holder opening. The needle shield gripper may be concavely curved in the region of the blocking portion or a region adjacent thereto, preferably immediately adjacent to the blocking portion. By way of the concave curvature, a smooth transition between an axially extending portion and the end surface may be facilitated along with the formation of a suitable blocking portion.

In an embodiment, the drug delivery device includes a housing. The housing may house a drive mechanism of the drug delivery device. The drive mechanism may include a spring. The drive mechanism may include a plunger which is biased by the spring to drive a delivery operation, e.g., by moving a stopper relative to the reservoir by means of the spring-driven plunger. The drug delivery device may be an autoinjector. The reservoir may be a prefilled syringe. The needle of the prefilled syringe may be covered by the needle shield. The needle shield may engage the pre-filled syringe, e.g., a syringe barrel thereof. The needle shield may be a rigid needle shield or a soft needle shield. The needle shield gripper may engage the needle shield. The rigid needle shield, as opposed a soft needle shield may have a rigid outer shell. Within the rigid outer shell a softer material may be provided which engages the needle.

In an embodiment, the needle shield removal assembly is a cap assembly for covering an end of the drug delivery device, e.g., a dispensing end. The content of the reservoir may be ejected from the drug delivery device via the dispensing end. The cap assembly may be removably connected to a housing or body of the drug delivery device such that it can be removed before an injection or delivery operation is performed. When the cap assembly is removed, the needle shield may be removed together with it. The cap assembly may cover an open end of the needle. The needle may be rigid.

In an embodiment, the needle is designed to pierce the skin of the patient using the drug delivery device.

In an embodiment, a method of assembling a needle shield removal assembly includes:
providing a gripper holder including an opening and a receiving space in communication with the opening
providing a needle shield gripper, the needle shield gripper including a gripper interface feature,
inserting the needle shield gripper into the opening,
guiding the needle shield gripper into the receiving space,
reducing or preventing mechanical contact of the gripper holder and the needle shield gripper during insertion of the needle shield gripper into the receiving space via the opening in at least one sensitive region of the gripper holder, and/or
securing the needle shield gripper within the gripper holder, e.g., via the connection interface described further above.

The sensitive region(s) may have been determined prior to the insertion of the needle shield gripper into the opening. The sensitive regions may be sensitive to the generation of flakes, scratches or scrapes during the insertion of the needle shield gripper into the receiving space. The insertion of the needle shield gripper into the receiving space, the gripper holder and/or the needle shield gripper may be adjusted or configured to reduce or prevent mechanical contact between the needle shield gripper and the gripper holder in the sensitive region(s), e.g., as described further above. The adjustment may include investigations of systems with needle shield grippers and gripper holders and identifying one or more sensitive regions. Thereafter, the gripper holder and/or the needle shield gripper may be modified or adjusted to reduce or prevent mechanical contact between the needle shield gripper and the gripper holder in the identified sensitive region(s).

In an advantageous embodiment, a needle shield removal assembly for a drug delivery device is provided, the assembly including:
a needle shield gripper configured to operatively engage a needle shield and to interlock with the needle shield, and
a gripper holder, wherein the needle shield gripper is secured to the gripper holder via a connection interface established between the gripper holder and the needle shield gripper, wherein the connection interface is formed by at least one holder interface feature of the gripper holder engaging at least one gripper interface feature of the needle shield gripper,
wherein the gripper holder has an opening, the opening being configured and dimensioned to receive the needle shield gripper and/or the needle shield, wherein the at least one holder interface feature is offset from the opening,
wherein the gripper holder defines a receiving space within which the needle shield gripper is received, wherein the receiving space has an axial extension, particularly directed away from the opening, and wherein, the needle shield gripper and the gripper holder are adjusted to one another to reduce or prevent mechanical contact of
   a) the gripper holder, in particular an inner surface thereof, and a bent or kinked region of the needle shield gripper and/or of
   b) the gripper holder, in particular an inner surface thereof, and an edge or corner of the needle shield gripper
      during insertion of the needle shield gripper into the receiving space via the opening in at least one sensitive region of the gripper holder which is angularly offset from the at least one holder interface feature.

As has been discussed above, the generation of scrapes or flakes can be reduced by considering the sensitive region.

In another advantageous embodiment, a needle shield removal assembly for a drug delivery device is provided, the assembly including:
a needle shield gripper configured to operatively engage a needle shield and to interlock with the needle shield, and
a gripper holder, wherein the needle shield gripper is secured to the gripper holder via a connection interface established between the gripper holder and the needle shield gripper,
   wherein the connection interface is formed by at least one holder interface feature of the gripper holder engaging at least one gripper interface feature of the needle shield gripper,
wherein the gripper holder has a holder opening, the holder opening being configured and dimensioned to receive the needle shield gripper and/or the needle shield, wherein the at least one holder interface feature is offset from the holder opening, e.g., axially and/or distally,
wherein the gripper holder defines a receiving space within which the needle shield gripper is received,
wherein the receiving space has an axial extension directed away from the holder opening, e.g., distally, and wherein,
the gripper holder has a radial recess or a radial opening in a section of the gripper holder which is axially and/or angularly offset from the at least one holder interface feature.

The recess or opening has been found to be particularly reliable to prevent the generation of scrapes or flakes.

In another advantageous embodiment, a needle shield removal assembly for a drug delivery device is provided, the needle shield removal assembly including:
a needle shield gripper configured to operatively engage a needle shield and to interlock with the needle shield, and
a gripper holder, wherein the needle shield gripper is secured to the gripper holder via a connection interface established between the gripper holder and the needle shield gripper,
   wherein the connection interface is formed by at least one holder interface feature of the gripper holder engaging at least one gripper interface feature of the needle shield gripper,
wherein the gripper holder has a holder opening, the holder opening being configured and dimensioned to receive the needle shield gripper and/or the needle shield,
wherein the holder interface feature is arranged to block movement of the needle shield gripper towards the holder opening by a blocking portion of the needle shield gripper abutting a surface of the holder interface feature facing away from the opening, and
wherein the blocking portion of the needle shield gripper is arranged in a region between an end surface of the needle shield gripper and an axially oriented portion of the needle shield gripper.

The blocking portion has been found to be particularly reliable to prevent the generation of scrapes or flakes.

Further features, advantages and advantageous embodiments of the present disclosure will become apparent from the detailed description of exemplary embodiments given below and the accompanying drawings. The exemplary embodiments should illustrate the concepts explained further above but are given by way of illustration only and are not intended to limit the present disclosure—neither the description nor the claims—to the embodiments which are discussed below. In the drawings:

### BRIEF DESCRIPTION OF THE FIGURES

FIG. 1 is a schematic perspective partial sectional view of an exemplary embodiment of an autoinjector.

FIGS. 2A and 3 are perspective views of an exemplary embodiment of a gripper formed of single piece of a sheet.

FIG. 2B is a cross section of an exemplary embodiment of a gripper.

FIG. 4 is a perspective view of an exemplary embodiment of a unitary sheet.

FIG. 5 is a cut-away view of an exemplary embodiment of a cap with an assembled gripper.

FIG. 6 is a cut-away view of an exemplary embodiment of a cap with an assembled gripper and an assembled protective needle shield.

FIGS. 7A and 7B illustrate another exemplary embodiment of a cap.

FIG. 8 schematically illustrates an embodiment of a gripper.

FIG. 9 shows a cap with scrapings or flakes being generated during the assembling of the cap and the gripper.

FIGS. 10A to 10D illustrate an embodiment of a gripper.

FIGS. 11A to 11C illustrate an embodiment of a cap.

FIGS. 12A and 12B illustrate an embodiment of the cap.

FIGS. 13A and 13B illustrate an embodiment of the gripper.

FIGS. 14A and 14B illustrate embodiments of the gripper.

FIGS. 15A and 15B illustrate an embodiment of the gripper.

FIG. 16 shows an embodiment with the gripper and the cap assembled to one another.

FIGS. 17A through 17G illustrate an embodiment of a cap and a gripper assembled to the cap.

FIGS. 18A through 18D illustrate another embodiment of the gripper and the cap.

### DETAILED DESCRIPTION

In the drawings, identical elements, elements of the same kind and identically acting elements may be provided with the same reference numerals.

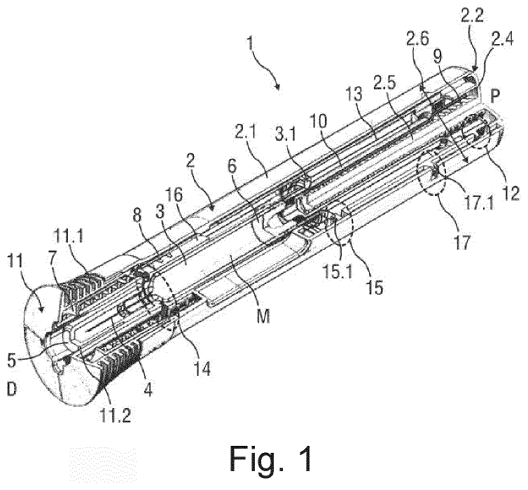

FIG. 1 is a schematic perspective partial sectional view of an exemplary embodiment of an assembled autoinjector 1.

The autoinjector 1 includes a housing 2. The housing includes a front part 2.1, e.g., sleeve shaped, and a rear part 2.2. The front and rear part are preferably secured to one another, e.g., irreleasably. Alternatively, the housing 2 may be formed as a one-piece housing (not shown).

The housing 2 is adapted to hold a reservoir, in particular a syringe 3, e.g., a glass syringe. The syringe 3 may be a pre-filled syringe containing a liquid medicament M. The syringe may have a needle 4 arranged at a distal end. In another exemplary embodiment, the syringe 3 may be a cartridge which includes the medicament M and engages a removable needle (e.g., by threads, snaps, friction, etc.). Alternatively, the cartridge may be brought into engagement with the needle during an injection operation. In the shown exemplary embodiment, the syringe 3 is held in the housing 2. The syringe 3 is supported, preferably at its proximal end, in the housing by a syringe support 15. Preferably, the syringe and/or the needle are stationary during the operation of the autoinjector. Although the embodiments are directed to autoinjectors the disclosed concepts relating to the needle shield removal may also apply to other types of delivery devices, i.e., non-autoinjectors.

The autoinjector 1 further includes a protective needle shield 5 that is coupled to the needle 4. For example, the protective needle shield 5 is removably coupled to the needle 4. The protective needle shield 5 may be a rubber needle shield (often termed: soft needle shield or SNS) or a rigid needle shield (in short: RNS) which is composed of rubber and/or a full or partial plastic shell. The needle shield 5 may engage the syringe and/or the needle. The needle shield may cover a tip of the needle.

A stopper 6 is arranged or provided for sealing the syringe 3 in a proximal direction P and for displacing the medicament M contained in the syringe 3 through the needle 4. That is to say, if the stopper is displaced distally relative to the syringe, medicament may be dispensed from the syringe. The stopper may seal the interior of the syringe proximally.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

The autoinjector 1 further includes a, e.g., sleeve-shaped, needle shroud 7. In an exemplary embodiment, the needle shroud 7 is telescopically coupled to the housing 2 and movable between an extended position relative to the housing 2 in which the needle 4 is covered and a retracted position relative to the housing 2 in which the needle 4 is exposed. Furthermore, a shroud spring 8 is arranged to bias the needle shroud 7 in a distal direction D with respect to the housing 2. Thus, the position in which the needle is covered may be the standard position of the needle shroud defined by the shroud spring.

A drive spring 9, e.g., in the shape of a, preferably helical, compression spring, is arranged within the housing 2, e.g., in a proximal part thereof, in particular the rear part 2.2. A plunger 10 serves for forwarding the force of the drive spring 9 to the stopper 6. In an exemplary embodiment, the plunger 10 is hollow and the drive spring 9 is arranged within the plunger 10, biasing the plunger 10 in the distal direction D against the rear part 2.2. In another exemplary embodiment, the plunger 10 may be a solid piece without a hollow and the drive spring 9 may engage a proximal end of the plunger 10. Likewise, the drive spring 9 could be wrapped around the outer diameter of the plunger 10 and/or extend into the syringe 3.

The autoninjector 1 further includes a plunger release mechanism 12. The plunger release mechanism 12 is arranged for preventing release of the plunger 10 prior to depression of the needle shroud 7 and for releasing the plunger 10 once the needle shroud 7 is sufficiently depressed. The plunger may be arranged to displace the stopper 6 during operation in order to eject or dispense medicament from the syringe 3. The needle shroud may therefore act as a trigger member which is moved to trigger an injection operation.

In an exemplary embodiment, the autoinjector 1 further includes at least one audible indicator 13 for producing an audible feedback for a user or patient indicating that medicament delivery is complete. In other words: The audible indicator 13 is adapted to indicate to a user or a patient that the full dose of medicament M was delivered. The audible indicator 13 is formed for example as a bistable spring and is held in the rear part 2.2.

To allow an accurate support of the syringe 3 during and/or after assembly, the autoinjector 1 includes a carrier 16 adapted to mount and hold the syringe 3 within the housing 2, e.g., in a forward or distal direction D. Due to manufacturing tolerances, syringes 3 may have variable lengths. Thus, a flange 3.1 of the syringe 3 protrudes from the carrier 16 in the proximal direction P. To support the axial position of the syringe 3 relative to the housing 2 after assembly, in particular during storage, transportation and normal use, the syringe support 15 includes one or more supporting beams 15.1 extend radially inwards or longitudinally to accommodate the variable length of the syringe 3 in an assembled state. The supporting beams 15.1 are adapted to axially bias the syringe 3 in the distal direction D within the housing 2 and to compensate the variable length of the syringe 3 in the distal direction D.

Furthermore, the autoinjector 1 includes a cap 11 that may be removably disposed at a distal end of the housing 2, in particular at a distal end of the front part 2.1. The cap 11 may be releasably connected to the housing, e.g., by a snap-fit or a threaded connection. The cap 11 may include outer grip features 11.1 for facilitating removal of the cap 11, e.g., by twisting and/or pulling the cap 11 off the housing 2. The cap 11 may further include a gripper 11.2 arranged to engage and grip the protective needle shield 5. The gripper 11.2 forms an inner grip element and is fixed to the cap 11. The cap 11 may be of plastic. The gripper 11.2 may be of metal.

The cap 11 is adapted to form part of a needle shield remover or removal assembly. For this purpose, the cap 11 and the gripper 11.2 are connected in such a manner that removal of the cap 11 together with the fixed gripper 11.2 from the autoinjector 1 removes the protective needle shield 5 from the needle 4. In other words: The gripper 11.2 is coupled to the cap 11 in a manner such that, when the cap 11 is removed, the protective needle shield 5 is also removed from the needle 4. Therefore, the cap assembly including the cap and the gripper secured to it is a needle shield removal assembly. The cap 11 acts as gripper holder.

Furthermore, a shroud pre-lock mechanism 14 is provided which is arranged and/or configured to prevent depression of the needle shroud 7 when the cap 11 is in place, thus avoiding unintentional activation of the autoinjector 1, e.g., if dropped, during shipping or packaging, etc.

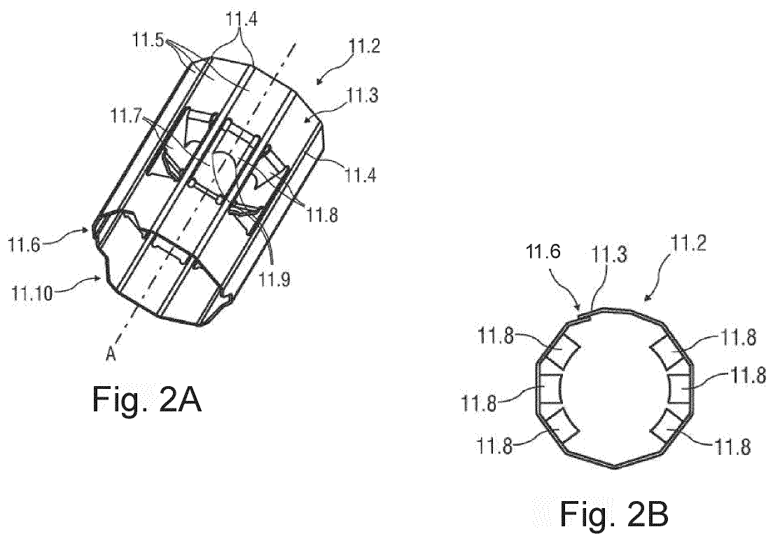

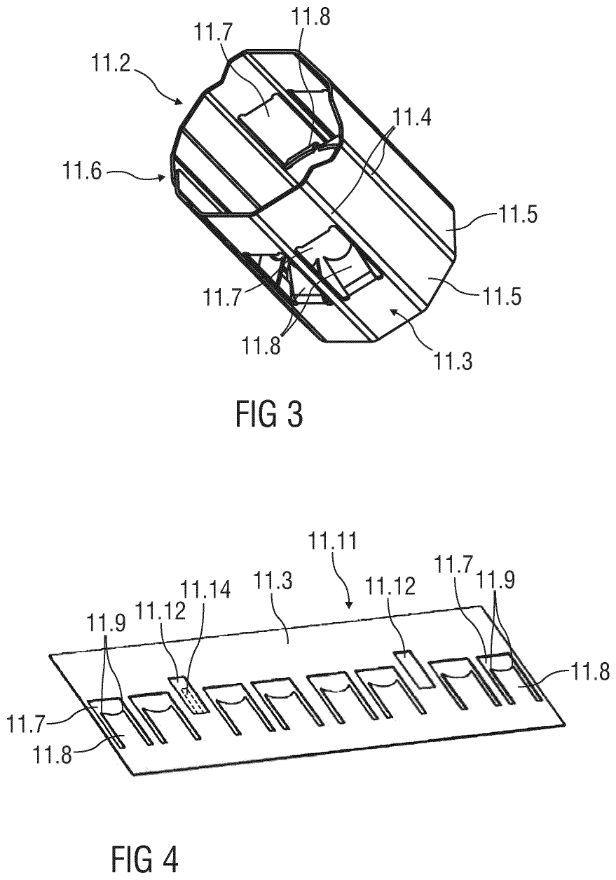

FIGS. 2A and 3 show an exemplary embodiment of a gripper 11.2 formed of a single piece, e.g., a sheet such as a metal sheet or a metal alloy sheet. FIG. 2B shows an exemplary embodiment of a cross section of the gripper 11.2.

The gripper 11.2 includes at least a gripper carrier or main body 11.3. The gripper carrier 11.3 is multiply bent or kinked along a plurality of longitudinal fold edges, kinks or bends 11.4 to form a plurality of carrier portions 11.5. The respective carrier portions may have or include a plan outer surface region. Furthermore, the gripper carrier 11.3 is bent or angled in such a manner that the outer carrier portions 11.5 are partly overlapping in an overlapping area 11.6. Hence, in the bent state the gripper carrier 11.3 has a pipe-form or tube-form with a polygonal cross section. The partly overlapping area 11.6 in the bent state of the gripper carrier 11.3 allows compensation of manufacturing tolerances of the gripper 11.2. The gripper carrier has free ends which may be arranged closed to the overlapping area 11.6

To grip the protective needle shield 5, more than one of the plurality of carrier portions 11.5 includes a cut-out or opening 11.7 from which a respective barb 11.8 is bent and projected inwards from the inner surface of the gripper carrier 11.3 and thus of the carrier portions 11.5. In the assembled state, the inwardly angled barbs 11.8 extend in the distal direction D of the autoinjector 1.

In the context of the present disclosure, “proximal” is used to designate a position which is further away or a direction pointing away from a dispensing end or an application site of the device or component of the device under consideration. “Distal” is used to designate a position which is closer to or a direction pointing towards a dispensing end or an application site of the device or component of the device under consideration

The barbs 11.8 are adapted to deflect and grip the protective needle shield 5 during assembly of the needle shield 5 into the autoinjector 1 and are adapted to further grip the needle shield 5 when the cap 11 is being removed from the autoinjector 1.

The barbs 11.8 are designed as hooks or have a prong-form. In particular, the barbs 11.8 are inwardly projected from the inner surface of the carrier portions 11.5 and include on its free end prongs 11.9. The prongs 11.9 are adapted to press onto or dig into the outer surface of the protective needle shield 5 and to form an interference fit during assembling or a positive and/or non-positive connection at least during removing of the protective needle shield 5. According to another aspect, the prongs 11.9 may be adapted to already dig into the outer surface of the protective needle shield 5 when the gripper 11.2 is being assembled to the protective needle shield 5 as described above. That is to say, a form-fit or positive fit may already be applied during the assembling process and not just after commencement of the cap removal process.

According to the present embodiment, the prongs 11.9 are configured as double spikes respectively arranged on each barb 11.8. This configuration is realized by a concave shape respectively between two prongs 11.9 per barb 11.8. Due to the concave shape and thus controlling the distance between the prongs 11.9, a penetration depth into the surface of the protective needle shield 5 may be limited. This is particularly important, when the needle shield is a rubber needle shield where penetration beyond a certain limit could impact sterility by accessing the needle 4.

Furthermore, for correct orientation of the gripper 11.2 during assembling within the cap 11, the gripper 11.2 includes an orientation element 11.10 indicating an assembling orientation. The orientation element 11.10 is designed as a tactile indicator or visual indicator or a combination of them. In particular, one of the front surfaces of the gripper carrier 11.3 is profiled, e.g., waved or pronged, and the other opposite front surface is even and flat. The orientation feature may be a cutout.

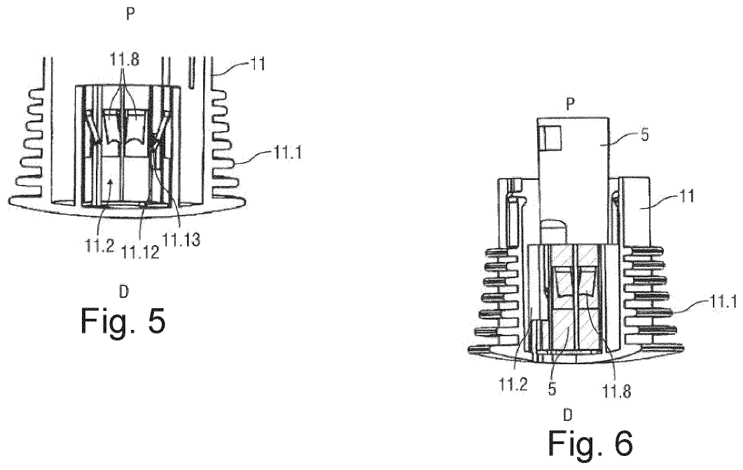

FIG. 4 shows an exemplary embodiment of a single piece of a sheet 11.11 which can form the gripper carrier 11.3. According to an aspect of the present disclosure, the gripper 11.2 is produced by the following steps:
providing the gripper carrier 11.3 in form of the sheet 11.11, e.g., a metal sheet such as a stamped or punched metal sheet;
forming a plurality of barbs 11.8 into the gripper carrier 11.3 by cutting, punching, stamping or die stamping;
multiply bending or kinking the gripper carrier 11.3 along a plurality of longitudinal fold edges or lines 11.4 to form a plurality of carrier portions 11.5 in such a manner that more than one of the plurality of carrier portions 11.5 includes a respective barb 11.8;
bending the barbs 11.8 in such a manner that the barbs 11.8 project from an inner surface of the associated carrier portion 11.5, e.g., as it is shown in FIGS. 2 and 3.

The sheet 11.11 may be a single piece of sheet metal which is cut, e.g., by punching or stamping, to form the cut-outs or openings 11.7 and barbs 11.8 in the cut-outs or openings 11.7.

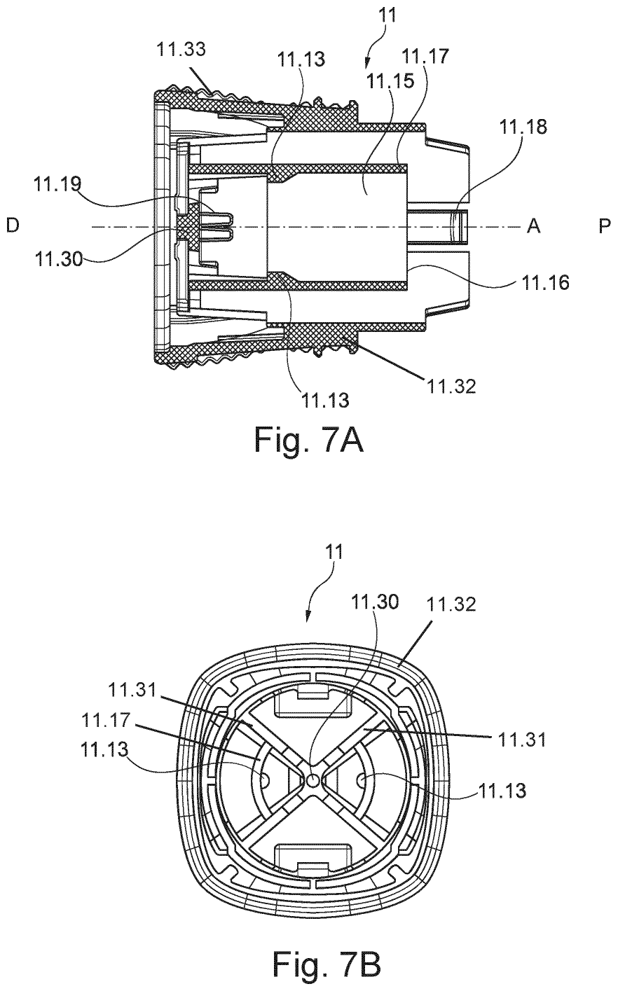

To retain the gripper 11.2 within the cap 11, the sheet 11.11 includes at least one retaining slot 11.12, which may be formed by cutting or punching into the gripper carrier 11.3. In the present embodiment, for example two retaining slots 11.12 are provided. The retaining slots 11.12 serve for retaining the gripper 11.2 within the cap 11, e.g., by holding lugs 11.13 of the cap 11 shown in FIG. 5. It is particularly advantageous, if the holding lugs 11.13 of the cap engage one of the openings 11.7, preferably of the barb in the middle of the respective group of barbs, to retain the gripper 11.2 in the cap 11. In this case, separate features like retaining slots 11.12 on the gripper to retain the gripper in the cap can be avoided. Rather, the opening or cut-out 11.7 serves as gripper interface feature engaging the holding lug. As the cap acts as gripper holder, the holding lugs may be the holder interface features discussed in the introductory section.

In an alternative embodiment, for retaining the gripper 11.2 within the cap 11, the gripper carrier 11.3 may include retaining lugs 11.14 (dotted line) and the cap 11 may include holding slots (not shown). In this case, the optional retaining lugs 11.14 and the barbs 11.8 of the gripper 11.2 are radially bent in opposite directions from the gripper carrier 11.3. In particular, the gripper carrier 11.3 is formed as an anchoring part or main part including the plurality of barbs 11.8 or claws inwardly directed away from the gripper carrier 11.3 and at an angle in the direction to a longitudinal axis A of the gripper 11.2 and bearing against the outer surface of the needle shield to grip the needle shield 5 wherein the retaining lugs 11.14 are outwardly bent and oriented at an angle away from the axis A and bearing against the inner cap surface of the cap 11 to grip the cap 11.

FIG. 5 is a cut-away view of an exemplary embodiment of a cap 11 with an assembled gripper 11.2 which is retained in the cap 11 by the holding lugs 11.13, engaging the retaining slots 11.12. The gripper 11.2 is fixed in the cap 11. The cap 11 includes the outer grip feature 11.1 which may be gripped by a user to remove the cap 11 from the autoinjector 1 wherein the cap 11 takes the gripper 11.2 and with the gripper 11.2 the protective needle shield 5 with it and, as a consequence, removes them together from the autoinjector 1 and thus from the needle 4. The barbs 11.8 are inwardly bent or angled and extend in the distal direction D to grasp the needle shield 5 in the assembled state or at the latest during removing of the protective needle shield 5 by removal of the cap 11.

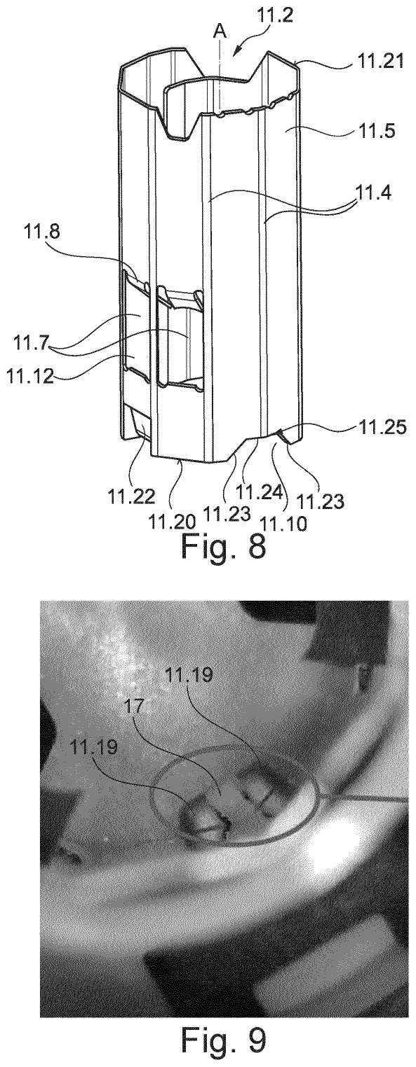

FIG. 6 is a cut-away view of an exemplary embodiment of a cap 11 with an assembled gripper 11.2 and a protective needle shield 5 assembled in the gripper 11.2 by an interference fit of the barbs 11.8 and the needle shield 5. The part of the needle shield 5 which is retained in the gripper 11.2 is shown in a transparent manner to show the interference fit of the barbs 11.8 and the needle shield 5.

As an alternative to the interference fit of the barbs 11.8 and the needle shield 5, the barbs 11.8 may be dug into the outer surface of the needle shield 5 to form a mechanical locking connection, e.g., a force-locking connection, form-fitting connection and/or friction-locking connection by interlocking structures, hooks and eyes or protrusions and undercuts, profiled surfaces, etc., at least during removing of the protective needle shield 5.

In an embodiment, the needle shield gripper 11.2 has at least one outer edge and/or inner edge which is defined by punching or cutting. Preferably, all outer edges and/or all inner edges of the needle shield gripper are defined by punching or cutting. This enables to form the needle shield gripper in a defined manufacturing step without significant subsequent modification steps to the part which has been manufactured, e.g., by punching. The part may be bent into the desired shape after punching or cutting as discussed for the metal sheet above. The manufacturing method, e.g., punching, may, however, generate sharp edges of the gripper as may the subsequent bending, wherein in the bent or kinked regions which are generated by bending, the effect is particularly pronounced, as in bent or kinked regions, the radius of curvature is particularly small with associated forces being particularly high when these regions—either the side faces defined by these regions or the corners at the ends of these regions—contact the wall of the cap/gripper holder.

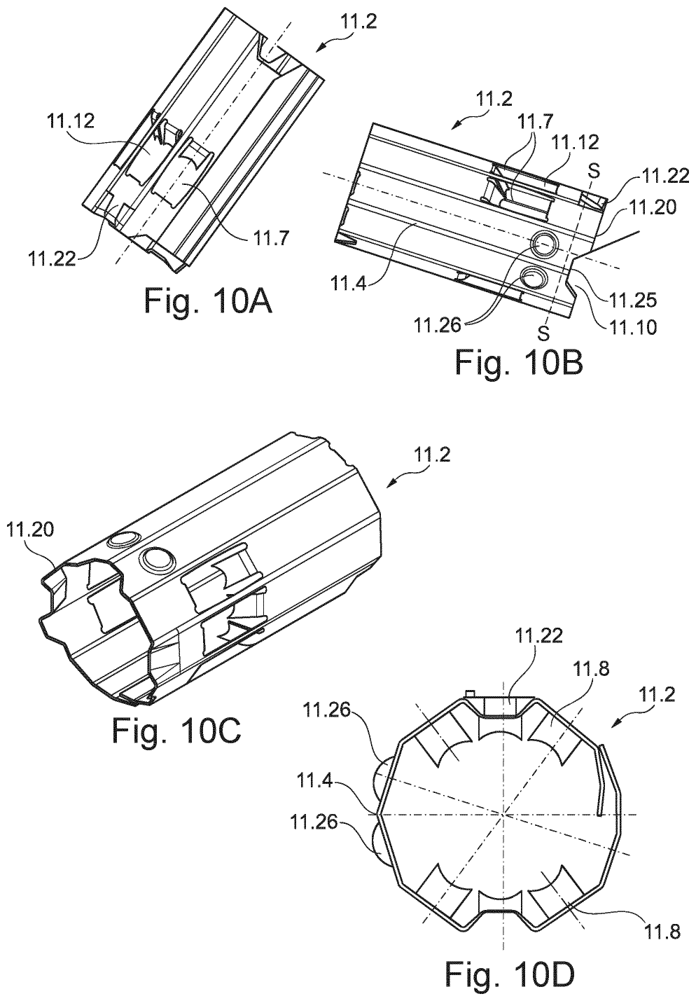

FIGS. 7A and 7B illustrate another exemplary embodiment of a cap 11. FIG. 7A shows a sectional view, whereas FIG. 7B shows a top view as seen from the distal end D of the cap. The cap essentially corresponds to the one discussed in connection with the previous embodiments. Therefore, features which have been discussed above already are not repeated here. Features which have not been discussed extensively in the previous embodiments include the receiving space 11.15, which is configured and/or dimensioned to receive the gripper 11.2 when it is introduced into the receiving space via an opening 11.16, e.g. a proximal opening or holder opening. When the gripper has been assembled to the cap, the gripper may be received in the receiving space in its entirety. The receiving space 11.15 may be defined by a tubular or sleeve-like portion 11.17 of the cap, which is dimensioned to receive the gripper 11.2 in its interior. In an embodiment, the portion 11.17 is inwardly offset from an interior surface of an outer wall 11.32 of the cap 11. The outer wall can be touched by the user at the outer surface to remove the cap and may be provided with grip features 11.33 at the outer surface thereof. At the end opposite of the opening 11.16, the receiving space 11.15 is delimited by an abutment feature 11.30. The abutment feature 11.30 includes, in the depicted embodiment, a plurality of webs 11.31. The webs 11.31 may be radially oriented. The webs may be evenly distributed in the circumferential direction. The webs may originate in the central region of the cap, when viewed from the opening in the distal direction and extend radially outwards. The webs 11.31 may extend towards an outer wall of the cap 11. The gripper 11.2 may abut the abutment feature 11.30 when the gripper is received in the receiving space. The retaining or holding lugs 11.13 are shown as holder interface features and are also designated as holder interface features in the following text. In the depicted embodiment, two holder interface features 11.13 are provided. They are oppositely disposed. If more than two holder interface features are provided, they may be evenly distributed in the circumferential direction as well. Moreover, at the proximal end of the cap 11, a housing connector 11.18 is provided. The housing connector 11.18 may establish a releasable connection, e.g., a snap-fit connection, with the housing 2. A plurality of housing connectors may be provided (not explicitly shown). The cap 11 further includes at least one or a plurality of gripper guide features 11.19. The gripper guide features 11.19 may protrude into the receiving space 11.15. The gripper guide features are arranged to interact with the orientation element or feature 11.10 of the gripper 11.2 particularly while introducing the gripper 11.2 into the receiving space 11.15. Due to the cooperation of the guide feature 11.19 and the orientation feature 11.10, the proper orientation of the gripper interface features, e.g., the retaining slots 11.12 or the cutouts or openings 11.7 which define the barbs 11.8, and the holder interface features 11.13 may be achieved. When oriented properly in the angular direction, the interface features are brought into engagement when advancing the gripper 11.2 axially within the receiving space 11.15.

The interface features 11.13 have, on the side facing the opening 11.16, a sloped section and on the side facing away from the opening a less sloped section, which, preferably, is more radially oriented than the sloped section. This facilitates advancing of the gripper 11.2 during assembly of the gripper 11.2 into the receiving space 11.15 with an associated elastic deformation in the radial direction. The gripper 11.2 may be elastically deformed already when it engages the section of the cap between the opening 11.16 and the interface features 11.13. When the gripper interface feature has passed the holder interface feature 11.13, it is securely retained in the receiving space 11.15 due to the radially oriented surface of the feature 11.13 preventing removal of the gripper 11.2.

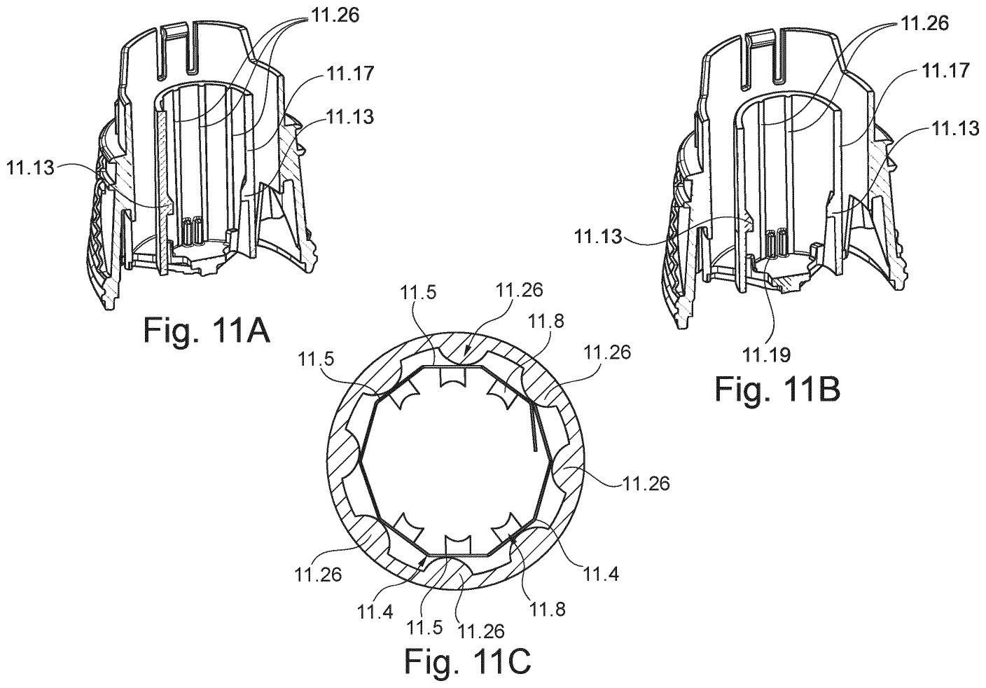

FIG. 8 schematically illustrates an embodiment of a gripper 11.2. The embodiment essentially corresponds to the ones previously described, which is why the description focusses on the differences. The gripper 11.2 has the same general shape as the previously described grippers 11.2 with the folded longitudinal edges 11.13, which form bent or kinked regions. However, a separate retaining slot 11.12 is not provided. Rather, the lugs or interface features 11.13 of the cap 11 shown in FIGS. 7A and 7B are designed to engage one of the openings 11.7, preferably the middle one of a group including three openings 11.7 where only two of the openings are shown in FIG. 8.

The gripper 11.2 has two opposite axial ends, the first or leading end 11.20 and the second or trailing end 11.21. The first end 11.20 is the one which is firstly introduced into the opening 11.15 during the assembling process. The gripper 11.2 has an oblique surface region 11.22 disposed at the leading end 11.20. The region 11.22 is inclined and directed away from the axis as seen from the first end 11.20. The region 11.22 may be designed to interact with the holder interface feature 11.13 which should engage the associated gripper interface feature 11.12. The oblique surface region 11.22 is aligned with the gripper interface feature 11.12 and/or the holder interface feature 11.13 which it should engage when it is assembled to the cap 11 angularly. Axially the oblique surface region 11.22 is offset from the gripper interface feature 11.12, which is may be formed by the opening 11.7 in the embodiment depicted in FIG. 8. When the holder interface feature 11.13 contacts the surface region 11.22 during insertion of the gripper 11.2 into the receiving space 11.16 and the gripper 11.2 is guided further into the opening 11.16, an elastic deformation of the gripper 11.2 in the radial direction may increase, e.g., until the gripper interface feature—opening 11.7/retaining slot 11.12—engages the holder interface feature 11.13. When the engagement is established, the elastic bias of the gripper 11.2 may be reduced, e.g., until the gripper abuts the cap 11.

In FIG. 8, the cutout forming the gripper orientation feature 11.10 is shown. The cutout has oblique side surfaces 11.23 which delimit the cutout angularly. Axially, the cutout is delimited by a surface 11.24 as seen in the axial direction away from the first end 11.20. The angular extension of the cutout may reduce with increasing distance from the first end 11.20. In other words, the cutout may taper towards the second end 11.21. The surface 11.24 delimiting the cutout axially may run perpendicularly to the axis A when seen in plan view or top view onto the cutout. The angle of the surfaces 11.23 relative to the axis may be less than 90°, e.g. 45 degrees or less, when seen in plan view or top view onto the cutout. The kinked or bent regions 11.4 may extend along the longitudinal direction of the gripper 11.2, preferably along the entire axial extension of the gripper 11.2.

Accordingly, at the leading end or leading edge, the kinks or bends 11.4 may define corners 11.25. The respective corner may be an angled region of the edge of the gripper 11.2. The edge or corner 11.25 may be oriented in the axial direction, i.e. face away from the opening 11.16. When guiding the gripper 11.2 depicted in FIG. 8 into the cap 11, e.g., the one depicted in FIGS. 7A and 7B, it has been found that the cap was scraped and or flakes of cap material tended to be generated. For example, flakes or scrapes of cap material were found at the bottom of the receiving space remote from the opening when the cap and the gripper had been assembled. Scraping or flake generation may decrease the user's confidence that the device is constructed properly and/or reduce the structural stability of the needle shield removal assembly due to the material which has been removed from the cap. Therefore, it is considered advantageous to reduce or prevent scraping or flake generation.

When investigating scraping or flake generation, it has been found that some regions are particularly sensitive for generating scrapes of flakes. These regions are interior regions of the cap and, in particular of the portion 11.17. Surprisingly, it has been found that regions which are angularly offset from the interface features 11.13, e.g. by 90° in case two interface features 11.13 are provided, are particularly sensitive as regards the generation of flakes or scrapes. One particular sensitive region was the one angularly aligned with the gripper guide feature 11.19, which may be angularly offset from the holder interface feature(s), e.g., by 90°. Alternatively or additionally, the region distally offset from the interface features 11.13 was particularly susceptible for damages of the cap caused by the gripper.

The presence of these regions may be due to the elastic deformation of the gripper 11.2 during the assembling process. Here, the gripper is at first is slightly elastically deformed, e.g., on account of the opening having a smaller diameter than the non-deformed gripper 11.2, before the gripper 11.2 engages the interface features 11.13. Then, the radial elastic deformation is increased. Accordingly, there may be considerably forces acting in the radial direction and those forces may tend to enlarge the diameter of the gripper in one or more regions which are angularly offset from the holder interface features 11.13. The scraping or flake generation was particularly pronounced when a bent or kinked region 11.4 and/or the associated corner 11.25 was arranged in the sensitive region.

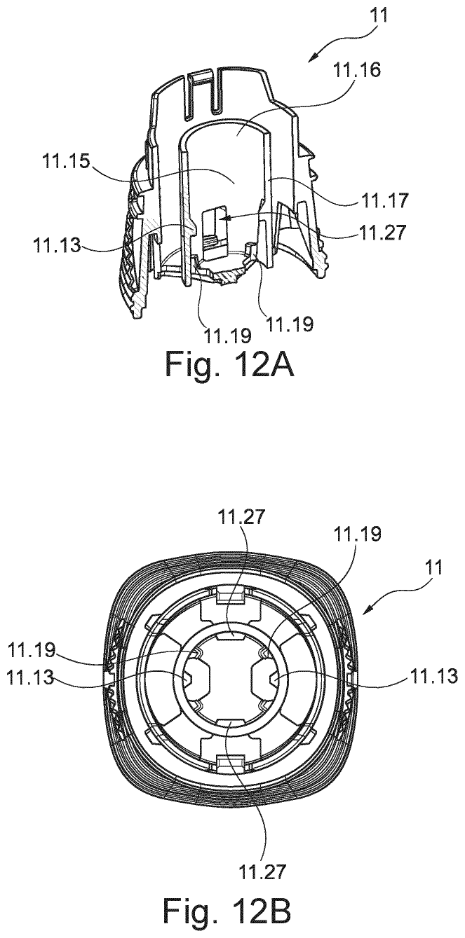

FIG. 9 illustrates the gripper guide feature 11.19 and a flake or scrape 17 which is generated or another kind of scraped material cap which occurred during the assembling process. The scrapes 17 were found predominantly in the region of the gripper guide feature 11.19 which is intended to cooperate with the orientation feature 11.10. In the embodiment depicted in FIG. 8, a kinked or bent region 11.4 which is angularly arranged in the region of the orientation feature 11.10 has been found to particularly contribute to the scraping, as it is angularly offset from the gripper interface feature 11.12/11.7 and/or the holder interface feature 11.13. The same may hold for corner 11.25 and/or a kinked or bent region arranged in the angular region of the other holder interface feature 11.13 depicted in FIG. 8. The main cause for the flake generation is, however, believed to be the elastic deformation occurring while the holder interface features engage a surface of the gripper 11.2 during the assembling. Particularly sensitive regions may be the regions into which the gripper 11.2 tends to deform if the diameter is reduced in the region including the gripper interface features/retaining slots 11.12, e.g., regions which are angularly offset from the gripper interface features.

The scraping or flake generation may be due to sharp edges formed during the manufacturing of the gripper 11.2, e.g., by punching or another process described previously, and/or by small radii of curvature in the kinked or bent region which are formed when bending the sheet to form the gripper 11.2.

In the following, some concepts are proposed which are suitable to reduce or prevent the scraping or flake generation in the gripper holder/cap 11, particularly in the sensitive region(s) thereof when assembling the gripper 11.2 to the cap 11.

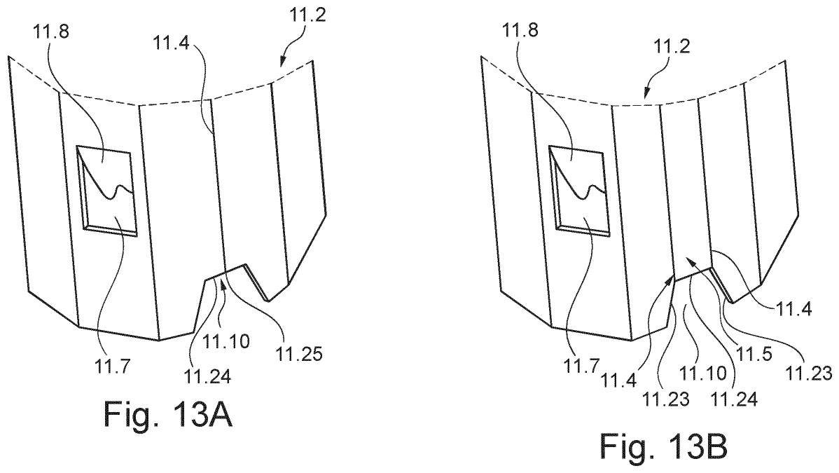

FIGS. 10A through 10D illustrate one concept where the gripper is modified, wherein FIGS. 10A through 10C show perspective views of the gripper 11 and FIG. 10D shows a sectional view, e.g. along the line S-S highlighted in FIG. 10B. The gripper 11.2 corresponds to the gripper disclosed and discussed in conjunction with FIG. 8 and the previous embodiments. Accordingly, only the modifications are discussed herein. In the shown embodiment, the gripper 11.2 is provided with radially oriented protrusions 11.26. The protrusions are arranged close to the angular and/or axial position of the corner 11.25 and/or the bent or kinked region 11.4 defining the corner 11.25, in particular the one(s) which is(are) arranged angularly between the two (opposite) gripper interface features/retaining slots 11.12 and/or the one which is to be disposed closest to the sensitive region. This corner 11.25 has been identified to be particularly likely to generate scrapes when contacting the (inner) wall of the cap 11, in particular of the portion 11.17. The respective protrusion 11.26 may be axially offset in a direction away from the opening and away from the holder interface features 11.13. The respective protrusion 11.26 may overlap angularly with the orientation feature or element 11.10. The respective protrusion 11.26 may axially overlap at least partly, e.g., only partly, with the opening 11.7 and/or the gripper interface feature/retaining slot 11.12.

The protrusions 11.26 may be formed by deep-drawing or embossing and can, therefore, be easily integrated into the manufacturing process of the gripper 11.2 as these methods are suitable methods for shaping metal parts or sheets.

In the region of the orientation feature 11.10, it is particularly advantageous, when the gripper 11.2 contacts the cap 11, as, otherwise, the mechanical cooperation with the gripper guide feature 11.19 might not be achieved. Lacking cooperation would impede the guiding functionality and increase the likeliness of not assembling the cap and the gripper properly, i.e., such that the connection interface between the interface features 11.12 and 11.13 is established. Consequently, in the region of the guide feature 11.10, it is particularly likely that scrapes or flakes occur. The protrusions may protrude by about 0.45 mm from the surface of the gripper 11.2. The protrusion may have a closed circumferential edge, e.g., a circular edge, on the outer surface of the gripper 11.2. The protrusions may have a general semi-spherical shape. The respective protrusion 11.26 may be confined to one surface of the gripper 11.2, e.g., the side surface. That is to say, when seen in top view onto the surface of the gripper, the respective protrusion 11.26 may be confined to that surface, preferably confined to one of the portions 11.5. As seen in top view onto a free end of the protrusion, the respective protrusion may have a circular or oval shape.

Moreover, as has been explained above, the orientation element or feature 11.10 is angularly offset from the holder interface feature 11.13 and/or the gripper interface feature 11.12 engaging the holder interface feature. This arrangement makes it likely that, due to elastic deformation, high contact forces between the gripper 11.2 and the gripper holder/cap 11 occur in this region. The protrusions 11.26 may be generated by embossing using a ball punch, e.g., a 2.0 millimeters diameter ball punch. The (maximum) diameter of the protrusion as seen in top view onto the free end may be less than or equal to 2 mm. The protrusions may protrude by less than 1 mm, preferably less than 0.8 mm or less than 0.7 mm, e.g. less than or equal to 0.5 mm from the outer surface, e.g. by 0.45 mm+/−0.05 mm. Alternatively or additionally, the respective protrusion protrudes by 0.3 mm or more, from the outer surface of the gripper 11.2. Protrusions which elevate from the outer surface by the respective height have proven to be particularly advantageous. For protrusions with a smaller height, e.g. 0.25 mm, the generation of scrapes or flakes was observed to occur with an increased likelihood. The protrusions 11.26 may be arranged such that they protrude radially beyond the bent or kinked region 11.4 arranged angularly between the protrusions 11.26 and/or beyond the corner 11.25 arranged angularly between the protrusions 11.26. Particularly, an enveloping curve, e.g., a circle, which runs through the free ends of the protrusions 11.26 may have a radial distance to the corner 11.25 and/or the bent or kinked region 11.4. In this way, the protrusions 11.26 may maintain a distance between the outer surface of the gripper 11.2 and the inner surface of the cap, particularly in that regions, where it matters the most, i.e., the sensitive region. In the depicted embodiment only two protrusions are provided which are associated with one bent or kinked region 11.4. However, it should be appreciated that more protrusions could be provided. Also, one or more protrusions could also be associated with different bent or kinked regions or corners. Although an arrangement with the bent or kinked region 11.4 angularly between two protrusions 11.26 may be advantageous, the arrangement of the protrusions is not restricted to such an arrangement. Likewise, the protrusions do not have to be arranged symmetrically relative to the kinked or bent region 11.4 as depicted. Rather, two protrusions could be arranged on one side of the bent or kinked region 11.4. Also just one protrusion could be provided. Moreover the number of protrusions on one side of the bent or kinked region may be greater than or equal to the number of protrusions on the other side of that bent or kinked region 11.4.

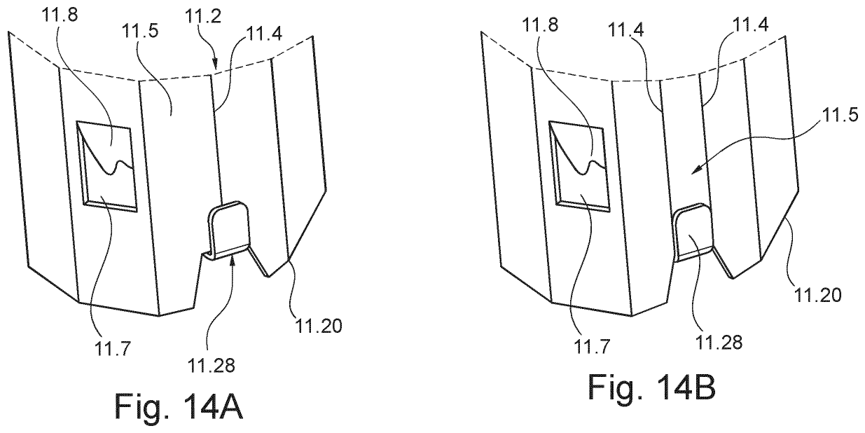

FIGS. 11A through 11C show another embodiment suitable to prevent scraping. FIGS. 11A and 11B are perspective sectional views illustrating two variants of the embodiment and FIG. 11C shows a sectional view taken perpendicularly to the longitudinal axis, e.g., for FIG. 11B.

In this embodiment, protrusions 11.26 are used as well to reduce or prevent mechanical contact. However, the protrusions 11.26 are not provided on the gripper 11.2 as in the previous embodiment but rather on the cap 11, e.g., on an interior surface of the portion 11.17. The protrusions 11.26 are directed radially inward. The protrusions 11.26 are designed as longitudinally extending ribs. The protrusions may extend along the entire extension of the portion 11.17 of the cap. The axial extension of the protrusions/ribs may be greater than or equal to 70% of the axial extension of the gripper 11.2. A plurality of protrusions 11.26, e.g., four or more, may be distributed, e.g., evenly, in the circumferential or angular direction (see FIG. 11A). As depicted in FIGS. 11A and 11B the protrusions 11.26 may be provided close to the sensitive region, e.g. the angular region of the gripper guide feature 11.19. In FIG. 11B the protrusions are only provided close to the sensitive region(s), whereas in FIG. 11A they are provided circumferentially. FIG. 11C shows a schematic view of the assembled gripper 11.2 for the variant in FIG. 11A. From this representation, it is clear, that the protrusions 11.26 contact the gripper 11.2 in a region between two adjacent bents or kinked regions 11.4. The contact regions may be plane outer surface regions or plane gripper portions 11.5 of the gripper 11.2. This concept has also proven to be suitable to prevent or reduce scraping or flake generation. Here, the variant in FIG. 11B was assessed as having a more decisive effect in respect of preventing scraping than the one in FIG. 11A, which also provided improvements. The protrusions 11.26 may protrude from an inner surface of the cap by less than or equal to 1 mm or less than or equal to 0.5 mm, such as by less than or equal to 0.3 mm. Alternatively or additionally, the protrusions 11.26 may protrude from the inner surface of the cap 11.2 by 0.3 mm or more. For example the height of the protrusion relative to the inner surface from which it protrudes may be 0.3 mm.

In an embodiment, the distance, preferably measured in the angular direction, i.e., the angular distance, between the respective protrusion 11.26—the protrusion being provided on the gripper holder/cap or on the gripper—and the associated sensitive region, the associated bent or kinked region, and/or the associated edge or corner of the needle shield gripper is less than or equal to one of the following values: 2 mm, 1.5 mm, 1 mm, 0.7 mm, 0.6 mm, 0.5 mm. In case of doubt, the distance may be measured from the boundary of the protrusion closest to the bent or kinked region and/or the edge or corner along the surface of the element having the protrusion until the angular position of the boundary of the bent or kinked region and/or the edge or corner is reached.

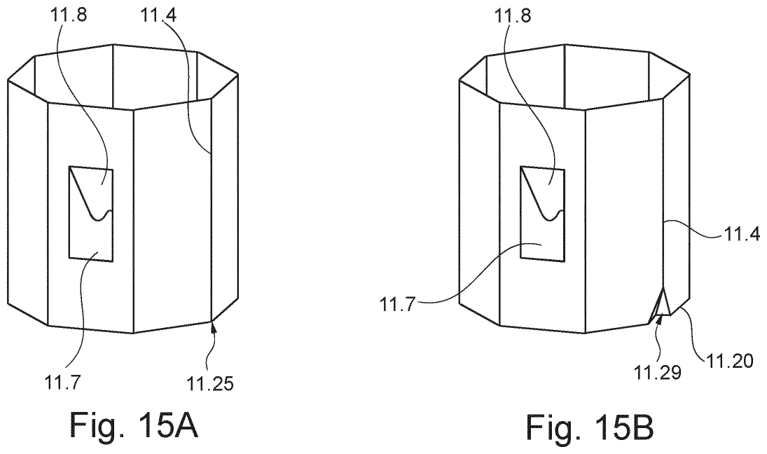

FIGS. 12A and 12B show another embodiment which is suitable to reduce or prevent scraping or flake generation. In this embodiment, in the sensitive region of the cap 11, an opening 11.27 is formed, in particular in the portion 11.17 which defines the receiving space 11.15. The opening 11.27 may extend radially through a section of the cap, e.g., the portion 11.17. The opening 11.27 may be defined during molding of the cap 11. The opening 11.27 may overlap axially with the interface feature(s) 11.13. The opening 11.27 may extend axially in the region distally or offset away from the opening 11.16 from the interface feature(s) 11.13, preferably in the entire region up to the end of the receiving space 11.15. The opening may overlap axially with the interface features 11.13. Using the opening 11.27 has been proven to be particularly advantageous as well with regards to avoiding scrapes or flakes. FIG. 12B shows a view on the cap 11 from the distal end and FIG. 12A shows a schematic section of the cap 11. It is noted that there is no guide feature 11.19 in the sensitive region due to the opening being provided in that region. The guide features 11.19 may be angularly offset from the opening 11.27 or the sensitive region as depicted. Therefore, despite of the opening in the sensitive region, there may still be a guiding interface established, which guides the interface features of the gripper and the cap 11/gripper holder into engagement during assembly.

Although the distal end of the cap 11 is shown to be open in some of the figures, it may be closed, e.g., by an appropriate lid part assembled to the cap 11 or by a closure unitary with the remainder of the cap 11. However, an open distal end facilitates the illustration of the embodiments.

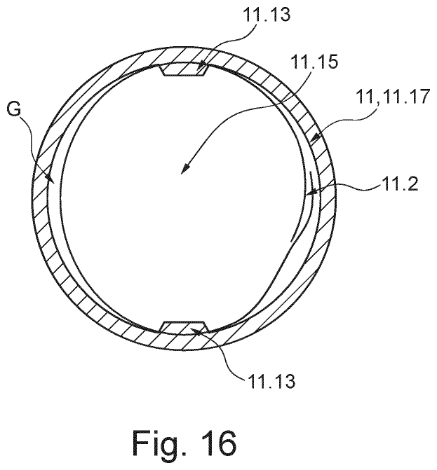

FIGS. 13A and 13B illustrate another embodiment. In FIG. 13A, a gripper 11.2 is shown as depicted in FIG. 8. Consequently, in the region of the orientation feature 11.10 a sharp corner 11.25 at the end of the bent or kinked region 11.4 is positioned, particularly within the surface 11.24 delimiting the cutout forming the orientation feature axially. The surface 11.24 may run perpendicularly or essentially perpendicularly relative to the axis A. FIG. 13B shows a possible solution to modify the embodiment of FIG. 13A to reduce scraping of the gripper holder. For doing so, the bent or kinked region 11.4 is moved away from the angular center of the cutout forming the orientation feature 11.10. For example, it may be re-positioned to be within the corner between the surfaces 11.23 and 11.24 and/or in the border region between the oblique surface 11.23 and the end surface 11.24. This arrangement may require an additional folding operation. A, preferably plane, carrier portion 11.5 may be positioned angularly in the region which has been identified as the sensitive region. This carrier portion may have been defined by an additional folding operation as compared to the gripper 11.2 shown in FIG. 13A. This concept may also be suitable to reduce scraping, as it removes the sharp edge or corner from the sensitive region to an angularly adjacent region. The angular width of the cutout, e.g., the maximum or minimum angular width of the cutout, forming the orientation element or feature 11.10 may be greater than or equal to the angular width of the sensitive region. Alternatively to the border region between the surfaces 11.23 and 11.24 the corner 11.25 may be positioned to be angularly overlapping with or be arranged in the oblique surface 11.23.

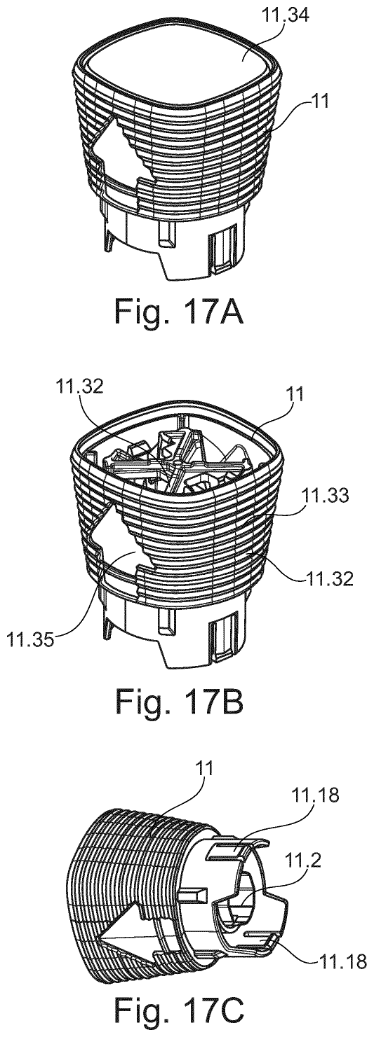

FIGS. 14A and 14B show another concept for reducing scraping. Here, in the relevant region of the gripper 11.2 a tab 11.28 is arranged radially outwardly offset from the bent or kinked region 11.4, particularly that region which would be arranged in the sensitive region of the portion 11.17 of the cap 11. In the original state of the gripper, i.e., when the tab has not yet been brought into the shown position, the tab may be oriented axially with the free end facing in the same direction as the leading end 11.20 of the gripper 11.2. The tab 11.28 may be folded or bent until it covers the outer surface of the gripper 11.2 and, particularly, the bent or kinked region 11.4. The free end of the tab 11.28 may then face in the same direction as the trailing end 11.21 of the gripper. FIG. 14B shows a combination of the concepts of FIG. 13B and FIG. 14A, where, in addition to moving the bent or kinked regions 11.4 angularly away from the sensitive region, the tab 11.28 is provided. This may facilitate manufacturing as the tab may then be more easily formed with a plane or curved surface which is less curved than the bent or kinked region. During assembling of the gripper to the cap, the tab with the plane surface may be arranged between the cap and the remainder of the gripper 11.2, thereby preventing contact between potentially scraping portions of the gripper and the cap. The outer surface of the tab 11.28 may be less kinked or curved than the bent or kinked region 11.4 and/or the corner 11.25.

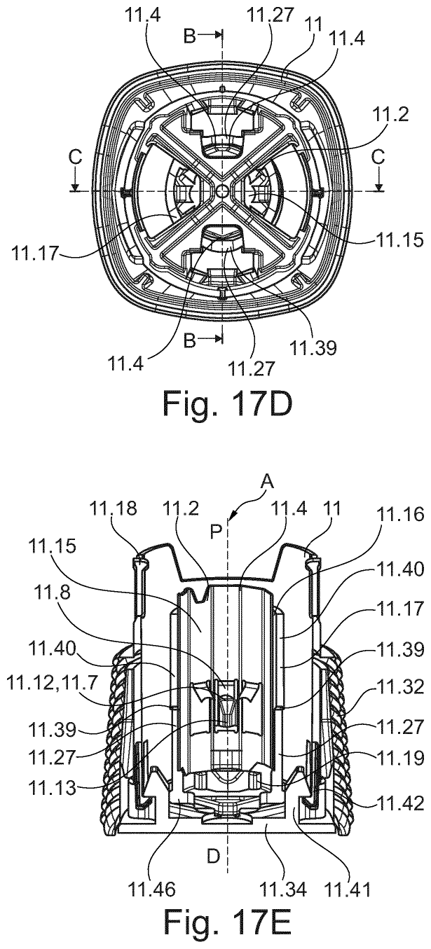

FIGS. 15A and 15B illustrate another concept of reducing or preventing scraping. FIG. 15A, again, schematically shows the gripper 11.2, e.g. the one depicted in FIG. 8. This gripper has a sharp corner 11.25, which would be disposed in the sensitive region of the cap 11. An engagement between this corner 11.25 and the (inner) surface of the cap 11 is avoided by providing an indentation 11.29. The indentation 11.29 is provided in the leading end 11.20 of the cap 11, particularly in the region where the sharp edge 11.25 and/or the kinked or bent region defining the corner 11.25 was positioned. The indentation 11.29 is oriented in the radial inward direction such that the edge 11.25 is inwardly offset from the inner surface of the cap 11, in particular the portion 11.17. Consequently, engagement of that surface and the edge is prevented. This will prevent scraping and flake generation during assembling as well.

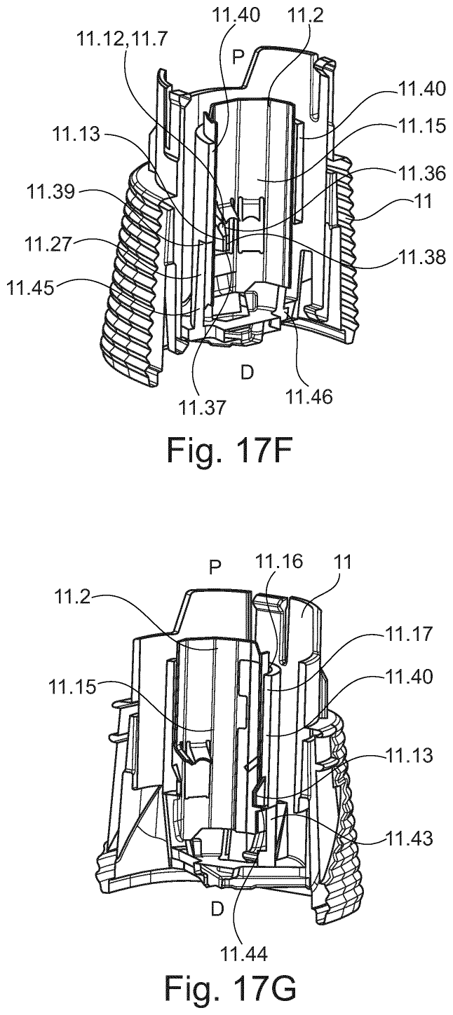

FIG. 16 illustrates another concept. This embodiment is also suitable to prevent scraping or flake generation during assembling of the gripper 11.2 to the cap 11. In this embodiment the gripper 11.2 is modified as compared to the one depicted in FIG. 8 in order to reduce or prevent scraping. In FIG. 16, the assembled state of the gripper 11.2 in the receiving space 11.15 of the cap 11 is illustrated. The remainder of the gripper 11.2 and the cap may, again, be constructed as shown in figures, 7A, 7B, and 8. As is apparent from FIG. 16, expediently in the potentially sensitive region(s), a gap G is formed between the gripper 11.2 and the cap 11. The gripper 11.2 is, in this embodiment, depicted as having a generally oval cross-section, i.e., a non-circular cross-section with a distinct longitudinal direction. The opening 11.16 may have a circular shape. The gripper 11.2 does not have to be formed oval in cross section. Rather, the gripper 11.2 could also be formed with a polygonal cross-section. In this case, the enveloping surface along the outer surface of the gripper may be oval or at least not rotationally symmetrical. The gripper 11.2 may have a distinct longitudinal main direction of extension, particularly in the radial direction. In the present embodiment, the main direction of extension may be aligned along the radial direction defined by the two opposite interface features 11.13. The gap G may be provided on just one side of the gripper 11.2 or on two, preferably opposite, sides, e.g., separated angularly by contact regions between the gripper and the cap. The gap G may take care that, when the gripper 11.2 is elastically deformed in the radial direction, the force acting on the cap 11 in that region where the gap is arranged is lowered as opposed to the situation when the gripper is arranged closer to the surface of the cap 11. Before the gripper 11.2 can contact the cap 11, the gap G has to be closed. The radial dimension of the gap G may be determined by the distance by which the interface features 11.13 protrude into the receiving space 11.15. Preferably, the cap 11 and the gripper 11.2 are adjusted such that the gap compensates the entire elastic deformation when the interface features 11.13 cause radial deformation of the gripper 11.2.

When the irregularly or rotationally asymmetrically shaped, e.g., oval, gripper 11.2 is inserted into the opening, it may still be elastically deformed by the contact with the wall delimiting the opening or the receiving space even before the interface features 11.13 engage with the outer surface of the gripper 11.2. However, the shape of the deformed gripper may still be such that the deformed gripper 11.2 has a distinct longitudinal axis and/or an oval enveloping curve as depicted in FIG. 16. Thus, when the interface features 11.13 are engaged, there is still a gap between the outer surface of the gripper and the inner surface of the cap which has to be closed before the gripper can contact the inner wall of the cap. The cross-section of the gripper may be polygonal or continuously curved, e.g., elliptical. The cross section may be different from the cross section of the opening and/or the region of the cap or the portion 11.17 between the opening 11.16 and the interface features 11.13, which may be regular or rotationally symmetrical. As in the previous embodiments, this concept may assist in preventing or reducing scraping or flake generation.

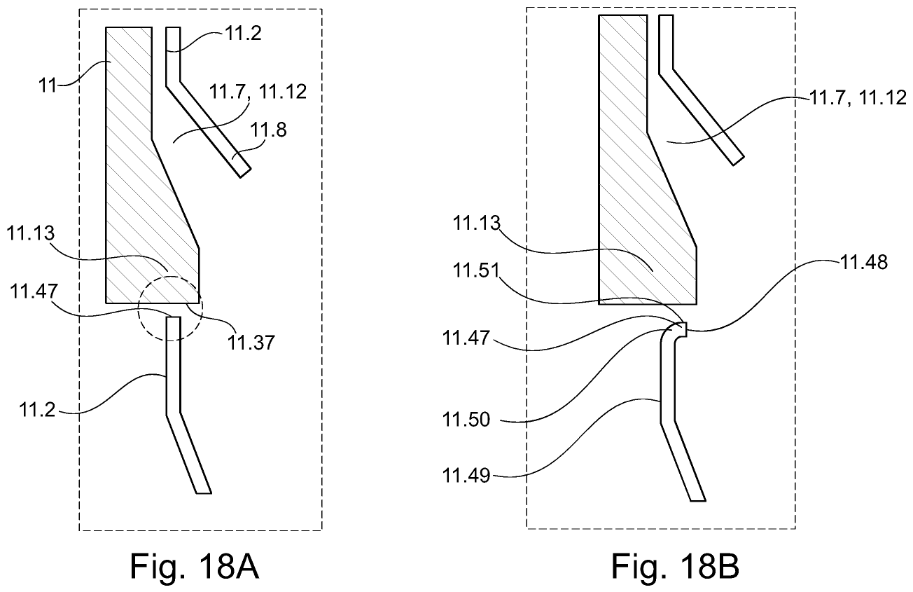

FIGS. 17A through 17G illustrate another embodiment of a cap 11 with an associated gripper 11.2 assembled to the cap 11 on the basis of different views. The cap 11 and the gripper 11.2 essentially correspond to the components described further above, such as in conjunction with FIGS. 2A through 8. Accordingly, the description associated with these figures does also apply for the present embodiment unless a particular difference is emphasized in the following text. Moreover, the gripper 11.2 may also have features discussed in conjunction with FIGS. 10A through 10D. However, as opposed to this embodiment, protrusions 11.26 are not provided in order to prevent or reduce contact between the cap and the gripper 11.2 in the sensitive region(s) which have been discussed further above. Rather, this embodiment uses a solution similar to the one discussed in conjunction with the embodiment depicted in FIGS. 12A and 12B. Accordingly, features disclosed in conjunction with this embodiment can also be used for the present embodiment unless a particular difference is emphasized in the following text. Vice versa, features disclosed in the present context can also be applied to other embodiments unless there is an incompatibility.

FIGS. 17A through 17C show perspective views of the cap 11. FIG. 17A shows the cap 11 with a distal end closed by way of a closure or lid 11.34. In FIG. 17B, the distal end of the cap 11 is open. For example, the webs 11.31 are visible. An indicator 11.35, e.g., a symbol, such as an arrow, is provided on the outer surface of the outer wall 11.32. The indicator 11.35 may be delimited by grip features 11.33. The indicator 11.35 may be recessed relative to the grip features 11.33. The indicator advantageously indicates the direction into which the cap has to be moved in order to be separated from the housing 2. Moreover, the housing connectors 11.18, e.g. resilient arms, are depicted.

FIG. 17D shows a top view onto the distal end of the cap 11. Here, the lid 11.34 is not connected to the cap 11. FIG. 17E shows a slightly perspective sectional view of the cap 11 basically taken along line B-B in FIG. 17D with the lid 11.34 connected to the cap 11. FIG. 17F shows a more oblique perspective sectional view of the representation in FIG. 17E without lid. The perspective sectional view depicted in FIG. 17G results from a section taken along line C-C in FIG. 17D.

As depicted in FIG. 17E, the gripper 11.2 is arranged within the receiving space 11.15 of the cap 11. The gripper 11.2 is arranged within the portion 11.17 of the cap 11. The portion 11.17 may be formed generally tube-like or sleeve-like. As depicted, the proximal end of the portion 11.17 is distally offset from a proximal end of the cap, e.g., a proximal end defined by the outer wall of the cap 11. Distally, e.g., close to the distal end of the cap 11, portion 11.17 may be connected to the outer wall 11.32 via the webs 11.31. A portion of the gripper 11.2 may protrude proximally from the portion 11.17. However, the portion 11.17 is expediently designed so as to receive at least the majority of the gripper, e.g., 80% or more of its total length. As depicted, the portion 11.17 is radially offset from an inner surface of the outer wall 11.32 of the cap 11. In this way, a free space is formed between the portion 11.17 and the outer wall 11.32. The gripper 11.2 may be in mechanical contact with one or more gripper guide features 11.19 with a distal surface, e.g., a distal end surface. These features may interact with the gripper 11.2 to preserve or achieve a defined rotational orientation of the gripper 11.2 relative to portion 11.17, e.g., when the gripper is introduced into the portion 11.17. As discussed already, the gripper 11.2 includes a plurality of longitudinal fold edges, kinks or bends 11.4.

The holder interface features or lugs 11.13 of the cap 11 are engaged with the gripper interface features (retaining slot 11.12 and/or cutout or opening 11.7). As depicted, the gripper interface feature may be one of the cutouts or openings 11.7 which defines the gripper features 11.8, e.g., barbs, which are designed to interlock with the needle shield. As depicted in FIG. 17E but visible more clearly in FIG. 17F, the holder interface feature 11.13 includes at its end facing towards the holder opening 11.16 and/or proximally an inclined region or sloped section 11.36 (only labelled in FIG. 17F). The, preferably plane, surface of that section or region may form or define an acute angle, e.g., less than 45°, with the longitudinal axis A. At its end facing away from the holder opening 11.16 and/or its distal end the holder interface feature 11.13 may be arranged to interact with a surface of the gripper 11.2, e.g., a surface delimiting the retaining slot 11.12 and/or the opening 11.7 distally. The end surface 11.37 of the holder interface feature 11.13 which is provided remote from the opening 11.16 or distally preferably defines or forms an angle with the axis A which is greater than the angle defined with the axis by region 11.36. For example, surface 11.37 may be oriented perpendicularly relative to the axis A. Region 11.36 and end surface 11.37 may be connected by a connection region 11.38 which extends substantially parallel to the axis A.

As discussed already in conjunction with the embodiment depicted in FIGS. 12A and 12B, radial openings 11.27 are provided. Openings 11.27 extend radially through the entire portion 11.17 and provide fluid communication between the receiving space and the free space arranged between the portion 11.17 and the inner surface of the outer wall 11.32 of the cap 11.

At least one edge, kink or bend 11.4 is arranged angularly in a position overlapping with the respective opening (see FIG. 17D). The edge, kink or bend 11.4 may protrude radially into the opening 11.27. In the radial opening 11.27 depicted in the upper half of FIG. 17D, a plurality of edges, kinks or bends 11.4 is arranged angularly and/or radially within the radial opening 11.27. There is a radial clearance between the cap 11 and the respective edge, kink or bend 11.4.

The openings 11.27 have a first axial end 11.39. The first axial end 11.39 may delimit the respective opening axially on the side of the opening 11.27 facing towards the holder opening 11.16 and/or proximally. Preferably, the first axial end is offset from the end of the holder interface feature 11.13 facing towards the holder opening—e.g., the proximal end such as the proximal end of the inclined region 11.36—or arranged axially overlapping with the inclined region 11.36 of the holder interface feature. In other words, the axial onset of the opening 11.27 may be before the axial onset of the holder interface feature as seen from the holder opening 11.16 towards the holder interface feature 11.13 along axis A. The openings 11.27 reduce the impact or a mechanical load transferred from the gripper 11.2 to the cap 11, especially in a section where the gripper is axially deformed when the section of the gripper distal to the retention slot 11.12 interacts with the holder interface feature 11.13 as has been described already. The respective opening 11.27 may be open at the second axial end 11.46 which is remote from the first axial end 11.39. That is to say, the opening 11.27 may be accessible from the distal end of the cap. This is easily recognizable in FIG. 17D, for example. The portion 11.17 defining the receiving space 11.15 has, e.g., on that side facing the holder opening 11.16, a section 11.40. Section 11.40 is advantageously circumferentially closed and/or has a tubular configuration. The section 11.40 may improve the structural integrity and stability of the portion 11.17.

It should be noted, that, aside from having a radial opening which protrudes radially through the entire portion 11.17, a recess, which is radially outwards delimited by the portion 11.17, would also reduce the generation of scrapes of flakes in the cap 11 when assembling the cap 11 and the gripper 11.2. However, the opening may have advantages with respect to manufacturing.

The axial offset between the proximal end of the holder interface feature 11.13 and the first axial end 11.39 may be less than the axial extension of section 11.40. The axial offset between the first axial end and the holder interface feature may be less than or equal to 2 mm, e.g. 1.5 mm. The axial extension of the holder interface feature 11.13 may be 2 mm. As an alternative to a non-zero offset of the opening in the proximal direction relative to the holder interface feature, the first axial end 11.39 may be arranged at the same axial position as the proximal end of the holder interface feature or within the inclined region 11.36.

The angular width of the radial openings 11.27 may increase in the distal direction and/or in the direction away from the holder opening. The angular width, e.g., the minimum angular width of the respective radial opening or the maximum angular width of the respective radial opening, may be greater than or equal to the angular extension of one carrier portion 11.5 of the gripper 11.2, e.g., the smallest or the largest angular extension of the carrier portions 11.5, if the dimensions of the carrier portions 11.5 in the angular directions vary.

Surfaces delimiting the radial opening in the angular direction, such as surface 11.45 shown in FIG. 17F may be oriented obliquely relative to a longitudinal axis A. The surfaces delimiting the opening angularly may be plane. As seen from the distal end D and along its axial extension the angular width of the radial opening 11.27 may reduce continuously up to its minimum at the first axial end 11.39.

As depicted in FIG. 17E, the lid 11.34 is connected to the cap 11, e.g., via a snap-fit connection which utilizes snap features 11.41 of the lid 11.34 cooperating with corresponding snap features 11.42. Snap features 11.42 may be flexible in the radial direction and the snap features 11.41 may be rigid such that the lid 11.34 may be connected to the cap by axially moving the lid onto the cap. Alternatively, the snap features 11.41 on the lid may be flexible radially and the snap features 11.42 may be rigid.

As depicted in FIG. 17G, further radial openings 11.43 (or recesses) are provided. The radial openings 11.43 are expediently provided at that side of the holder interface features 11.13 which is remote from the holder opening 11.16. The respective opening may be provided in the portion 11.17. A respective opening 11.43 may be arranged distally from the respective holder interface feature 11.13, e.g., angularly aligned with this feature. On the side facing towards the holder opening 11.16 and/or proximally the opening 11.43 may be delimited axially by a surface 11.44. The surface 11.44 may simultaneously form the end surface 11.37 of the holder interface feature 11.13, e.g., the distal end. The radial opening 11.43 may provide advantages during manufacturing of the assembly including the cap 11 and the gripper holder 11.2. For example, it may provide space to secure the cap 11 in a mount or to apply a tool to handle the cap 11 while introducing the gripper holder 11.2.

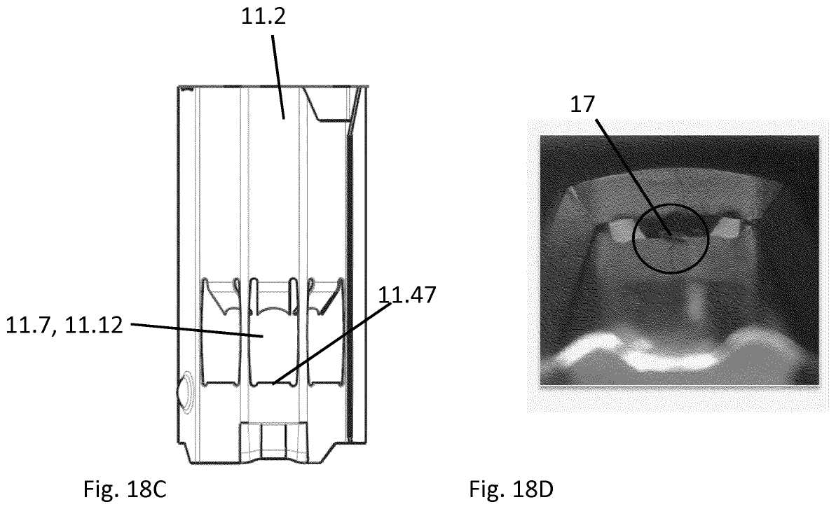

FIGS. 18A through 18D illustrate another embodiment of a needle shield removal assembly. When investigating the solution discussed in conjunction with FIGS. 17 and 12 and similar solutions, it has been discovered that the generation of scrapes of flakes has been reduced considerably. However, in rare cases, residual scrape or flake generation could still be observed. The observed flake or scrape generation usually occurred in the region where the holder interface feature 11.13 (holding lug) of the cap/gripper holder 11 engaged the gripper interface feature (retaining slot 11.12 or opening 11.7). This region is shown in the schematic representation in FIG. 18A. FIG. 18A shows the holder interface feature 11.13 and the gripper 11.2 with the needle shield interlock features or barbs 11.8. The region where the flake generation was observed is encircled. This region is arranged distally from a distal end surface 11.37 of the holder interface feature 11.13. FIG. 18C shows an embodiment of the gripper 11.2, where the region where flake generation was still observed is in the region with reference numeral 11.47. Reference numeral 11.47 designates a, preferably flexible, tab or tongue which is angularly delimited by axially extending cutouts. The tab or tongue is preferably flexible with respect to the remainder of the gripper 11.2. The axial extension of the tab or tongue may be less than 20% of the axial extension of the opening, e.g., less than 2 mm. This region was identified as contributing to the remaining scrape of flake generation as is evident from FIG. 18D which shows a picture taken from the gripper 11.2 where a flake 17 is shown in the encircled region.

FIG. 18B illustrates a solution for avoiding flake or scrape generation in the region encircled in FIG. 18A. Specifically, the tab or tongue 11.47 is bent or curved inwardly such that a free end 11.48 of the tab or tongue 11.47 faces in the radial inward direction, e.g., towards the axis. In this way, contact between the gripper holder 11 and in particular of the holder interface feature 11.13 with sharp surface regions or edges defined in the region of the (free) end 11.48 of the tab or tongue 11.47 may be avoided. Accordingly, as seen from a distal axially extending region or portion 11.49 of the gripper 11.2, the gripper 11.2, particularly in the region which delimits the opening which is engaged by the holder interface feature, may change its orientation via a transition region 11.50 to a radial orientation. The change may end when the end surface of the free and 11.48 faces in the radial direction. However, further bending is also possible such that the end is, for example, oriented distally resulting in an inverted U-like shape of the gripper 11.2 in the relevant portion (not shown in this embodiment). The blocking portion 11.51 interacting with the holder interface feature 11.13 to prevent removal of the gripper 11.2 from the gripper holder 11 is between the axial portion 11.49 and the end 11.48 as seen along the extension of the gripper 11.2. Preferably, the blocking portion 11.51 is within the transition region 11.50 within a radially oriented region of the gripper. The blocking portion may be directly adjacent to the end 11.48, e.g. within 2 mm or less or 0.5 mm or less from the end 11.48 as seen along the surface of the needle shield gripper 11.2.

It should be noted that every gripper interface feature may be delimited axially by an according blocking portion.

Bending the tab or tongue may be comparatively easily implemented in the manufacturing process and does not require excessive adjustments such as new cutting or punching processes being established, for example.

In an embodiment of a method of assembling a needle shield gripper, a gripper structure may be formed, e.g., from a metal sheet. The gripper structure can be selectively deformed, e.g., bent, or treated in a region to form the blocking portion, e.g., the blocking portion which is specified further above. Forming the blocking portion may include deforming the region of the gripper structure such that a portion of the gripper structure including the end surface, e.g., the surface of end 11.48, is displaced out of a plane which is delimited by one, more, or all of the remaining boundary surfaces of a gripper structure opening in the gripper structure, where the end surface, before the portion was displaced, is arranged in the gripper structure opening, e.g., a sheet-like structure as depicted in FIG. 4. The displaced portion may define the blocking portion, e.g., a curved or rounded portion. The needle shield gripper may be shaped from the gripper structure, e.g., by bending a sheet-like gripper structure into a desired shape for the needle shield gripper The finished needle shield gripper includes the blocking portion, which may be formed either before or after the (re-)shaping process performed with the gripper structure. The needle shield gripper may then be guided into the gripper holder or cap 11 as described previously already to form the needle shield gripper assembly.

With a solution including the blocking portion, the risk of flake or scrape generation is further reduced which is why the according embodiment is particularly suitable, preferably, but not exclusively, in combination with the solutions discussed in FIGS. 12 and 17. As opposed to the solution discussed in conjunction with FIGS. 12 and 17 which rely on measures taking place at an angular offset relative to the holder interface feature, the solution discussed in conjunction with FIG. 18 relies on a measure which occurs in the region angularly overlapping with the holder interface feature. We note that further features, which are described herein with respect to the needle shield assembly, also apply to this embodiment. Also, other embodiments could also use the features of this embodiment.

It should be noted that features which are disclosed above in conjunction with different aspects and embodiments can be combined with one another regardless of whether they are contained in the description of the exemplary embodiments or in the introductory or summary section of the present disclosure. Moreover, features, which are discussed only in the introductory section of the disclosure, should also be considered as being disclosed in the section discussing the exemplary embodiments and vice versa.

The scope of protection is not limited to the examples given herein above. Any invention disclosed herein is embodied in each novel characteristic and each combination of characteristics, which particularly includes every combination of any features, which are stated in the claims, even if this feature or this combination of features is not explicitly stated in the claims or in the examples.

### REFERENCE NUMERALS

1 autoinjector
2 housing
2.1 front part
2.2 rear part
2.4 proximal end
3 syringe
3.1 flange
4 needle
5 needle shield
6 stopper
7 needle shroud
8 shroud spring
9 drive spring
10 plunger
11 cap
11.1 grip feature
11.2 gripper
11.3 gripper carrier
11.4 longitudinal fold edges
11.5 carrier portion
11.6 overlapping area
11.7 cut-out/opening
11.8 barb
11.9 prong
11.10 orientation element
11.11 sheet
11.12 retaining slot
11.13 holding lug
11.14 retaining lug
11.15 receiving space
11.16 opening
11.17 portion
11.18 housing connector
11.19 gripper guide feature
11.20 first/leading end
11.21 second/trailing end
11.22 oblique surface region
11.23 surface
11.24 surface
11.25 edge/corner
11.26 protrusion
11.27 opening
11.28 tab
11.29 indentation
11.30 abutment feature
11.31 web
11.32 outer wall
11.33 grip features
11.34 lid
11.35 indicator
11.36 inclined region
11.37 surface
11.38 region
11.39 axial end
11.40 section
11.41 snap feature
11.42 snap feature
11.43 opening
11.44 surface
11.45 surface
11.46 axial end
11.47 tab or tongue
11.48 free end
11.49 axial portion
11.50 transition region
11.51 blocking portion
12 plunger release mechanism
13 audible indicator
14 shroud pre-lock mechanism
15 syringe support
15.1 supporting beam
16 carrier
17 scrape/flake
A Longitudinal axis
D distal direction
G gap
M medicament
P proximal direction

1-41. (canceled)

42. A needle shield removal assembly for a drug delivery device, the needle shield removal assembly comprising:
a needle shield gripper configured to operatively engage a needle shield and to interlock with the needle shield; and
a gripper holder, wherein the needle shield gripper is secured to the gripper holder via a connection interface established between the gripper holder and the needle shield gripper, wherein the connection interface is formed by at least one holder interface feature of the gripper holder engaging at least one gripper interface feature of the needle shield gripper;
wherein the gripper holder has a holder opening, the holder opening being configured and dimensioned to receive the needle shield gripper and/or the needle shield, wherein the at least one holder interface feature is offset from the holder opening;
wherein the gripper holder defines a receiving space within which the needle shield gripper is received;
wherein the receiving space has an axial extension directed away from the holder opening; and
wherein the gripper holder has a radial recess or a radial opening in a section of the gripper holder which is axially and/or angularly offset from the at least one holder interface feature.

43. The needle shield removal assembly of claim 42, wherein the receiving space is delimited by a portion of the gripper holder, and wherein the portion of the gripper holder comprises a section with a cylindrical surface provided between the holder opening and the holder interface feature.

44. The needle shield removal assembly of claim 43, wherein the portion of the gripper holder is radially inwardly offset from an inner surface of the gripper holder.

45. The needle shield removal assembly of claim 42, wherein the radial recess or radial opening is connected to the receiving space and extends outwardly as seen from the receiving space into or through the gripper holder or a portion thereof.

46. The needle shield removal assembly of claim 42, wherein the at least one holder interface feature protrudes into the receiving space.

47. The needle shield removal assembly of claim 42, wherein the gripper holder is a unitary component.

48. The needle shield removal assembly of claim 42, wherein an angular width of the radial recess or radial opening varies as seen along an axial extension of the radial recess or radial opening.

49. The needle shield removal assembly of claim 42, wherein a first axial end of the radial recess or radial opening which faces towards the holder opening is axially offset from the holder opening, and wherein a second axial end of the radial recess or radial opening is axially offset from the at least one holder interface feature in a direction away from the holder opening.

50. The needle shield removal assembly of claim 49, wherein the second axial end of the radial recess or radial opening which is remote from the first axial end is an open axial end, or
wherein the first axial end of the radial recess or the radial opening is associated with an axial position of the holder interface feature, or
wherein the first axial end of the radial recess or radial opening is arranged closer to the holder opening than an end of the at least one holder interface feature facing towards the holder opening, or
wherein the first axial end of the radial recess or radial opening and an end of the at least one holder interface feature facing towards the holder opening are axially aligned.

51. The needle shield removal assembly of claim 42, wherein the at least one holder interface feature comprises an inclined region at an end of the holder interface feature facing towards the holder opening.

52. The needle shield removal assembly of claim 42, wherein the needle shield gripper comprises at least one kinked or bent region which overlaps with the radial opening or radial recess as seen in the angular direction.

53. The needle shield removal assembly of claim 52, wherein an angular width of the radial recess or radial opening is greater than an angular width of the kinked or bent region of the needle shield gripper.

54. The needle shield removal assembly of claim 42, wherein the holder interface feature is arranged to block movement of the needle shield gripper towards the holder opening by a blocking portion of the needle shield gripper abutting a surface of the holder interface feature facing away from the opening.

55. The needle shield removal assembly of claim 54, wherein the blocking portion of the needle shield gripper is arranged in a region between an end surface of the needle shield gripper and an axially oriented portion of the needle shield gripper, or
wherein the blocking portion is arranged in a region of a tab or tongue of the gripper holder, or
wherein the end surface faces in the radial inward direction, or
wherein the end surface is a surface defined by cutting or punching.

56. The needle shield removal assembly of claim 54, wherein the blocking portion is arranged in a transition region of the needle shield gripper, or
wherein, in the transition region, the needle shield gripper changes the orientation from a predominantly axial orientation to a predominantly radial orientation, or
wherein the needle shield gripper is concavely curved in the region of the blocking portion or a region adjacent to the blocking portion, or
wherein the gripper interface feature is an opening delimited axially by the blocking portion, or
wherein the needle shield gripper has at least one needle shield interlock feature, the needle shield interlock feature being adapted to interlock with the needle shield, wherein
a) the blocking portion is angularly aligned with one of the needle shield interlock features and/or
b) the needle shield interlock feature protrudes radially beyond the end surface.

57. The needle shield removal assembly of claim 42, wherein the needle shield gripper is elastically deformable in the radial direction, or
wherein the needle shield gripper comprises an oblique surface region which is arranged at an axial position offset from the holder interface feature in a direction away from the opening, and wherein the oblique surface region angularly overlaps with the at least one holder interface feature, or
wherein the needle shield gripper has a plurality of needle shield interlock features, each of the plurality of needle shield interlock features being adapted to interlock with the needle shield, wherein the at least one holder interface feature is angularly aligned with one of the plurality of needle shield interlock features.

58. The needle shield removal assembly of claim 42, wherein the gripper holder comprises a radially extending recess or a radial opening in a sensitive region to prevent or reduce mechanical contact with the needle shield gripper in the sensitive region, or wherein the needle shield gripper comprises a plurality of bent or kinked regions, wherein a plane surface region or a less bent surface region of the needle shield gripper is arranged between two adjacent bent or kinked regions, and wherein the at least one sensitive region is arranged angularly between two adjacent bent or kinked regions.

59. A drug delivery device comprising the needle shield removal assembly of claim 42 and a reservoir, the reservoir comprising a drug, wherein the drug delivery device further comprises a needle and a needle shield, the needle being at least partly covered by the needle shield, wherein the needle shield gripper engages the needle shield such that the needle shield can be removed from the needle using the needle shield removal assembly, and wherein the needle shield removal assembly is a cap assembly of the drug delivery device.

60. A needle shield removal assembly for a drug delivery device, the needle shield removal assembly comprising:
a needle shield gripper configured to operatively engage a needle shield and to interlock with the needle shield; and
a gripper holder, wherein the needle shield gripper is secured to the gripper holder via a connection interface established between the gripper holder and the needle shield gripper, wherein the connection interface is formed by at least one holder interface feature of the gripper holder engaging at least one gripper interface feature of the needle shield gripper;
wherein the gripper holder has a holder opening, the holder opening being configured and dimensioned to receive the needle shield gripper and/or the needle shield;
wherein the holder interface feature is arranged to block movement of the needle shield gripper towards the holder opening by a blocking portion of the needle shield gripper abutting a surface of the holder interface feature facing away from the opening; and
wherein the blocking portion of the needle shield gripper is arranged in a region between an end surface of the needle shield gripper and an axially oriented portion of the needle shield gripper.

61. A needle shield removal assembly for a drug delivery device, the needle shield removal assembly comprising:
a needle shield gripper configured to operatively engage a needle shield and to interlock with the needle shield; and
a gripper holder, wherein the needle shield gripper is secured to the gripper holder via a connection interface established between the gripper holder and the needle shield gripper, wherein the connection interface is formed by at least one holder interface feature of the gripper holder engaging at least one gripper interface feature of the needle shield gripper;
wherein the gripper holder has an opening, the opening being configured and dimensioned to receive the needle shield gripper and/or the needle shield, wherein the at least one holder interface feature is offset from the opening;
wherein the gripper holder defines a receiving space within which the needle shield gripper is received, wherein the receiving space has an axial extension directed away from the opening; and
wherein the needle shield gripper and the gripper holder are adjusted to one another to reduce or prevent mechanical contact of
a) the gripper holder and a bent or kinked region of the needle shield gripper and/or of
b) the gripper holder and an edge or corner of the needle shield gripper during insertion of the needle shield gripper into the receiving space via the opening in at least one sensitive region of the gripper holder which is angularly offset from the at least one holder interface feature.
